(19) United States
(12) Patent Application Publication
(10) Pub. No.: US 2022/0389374 A1
(43) Pub. Date: Dec. 8, 2022

(54) USE OF FUNCTIONALIZED AND NON-FUNCTIONALIZED ECMS, ECM FRAGMENTS, PEPTIDES AND BIOACTIVE COMPONENTS TO CREATE CELL ADHESIVE 3D PRINTED OBJECTS

(71) Applicant: Lung Biotechnology PBC, Silver Spring, MD (US)

(72) Inventors: Aman Kaur, Manchester, NH (US); Victor Hernandez, Cambridge, MA (US); Barbara Nsiah, Manchester, NH (US); Isabel Arias, Manchester, NH (US); Luis Alvarez, Lexington, MA (US)

(73) Assignee: Lung Biotechnology PBC, Silver Spring, MD (US)

(21) Appl. No.: 17/738,694

(22) Filed: May 6, 2022

Related U.S. Application Data
(60) Provisional application No. 63/185,293, filed on May 6, 2021.

(51) Int. Cl. C12N 5/00; B33Y 10/00; B33Y 80/00; A61L 27/36; A61L 27/24; A61L 27/18

(52) U.S. Cl. CPC C12N 5/0068; B33Y 10/00; B33Y 80/00; A61L 27/3633; A61L 27/24; A61L 27/18; C12N 2533/90; C12N 2533/54

(57) ABSTRACT

Embodiments of this disclosure relate to bioinks and bioink compositions. These bioinks may be 3D printed into a hydrogel. The printed hydrogel may support primary cell and induced pluripotent stem cell attachment, proliferation, and spreading. Compounds in the bioink may be modified to incorporate chemical functionality, such as by chemical synthesis means. Incorporating chemical functionality may allow the incorporation of modified material as a component in the bioink. The modifications may allow chemical conjugation of a desired component. The desired component may maintain its cell interactive feature to aid in cell attachment and proliferation. Such incorporation may allow modulation of the bioprinted object's mechanical properties without interfering with cell adhesion.

### CROSS-REFERENCE TO RELATED APPLICATIONS

This application claims priority to U.S. Provisional Application No. 63/185,293, filed May 6, 2021, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Jul. 21, 2022, is named 080618-2084 SL.txt and is 8,683 bytes in size.

### BACKGROUND

Compositions, including hydrogels, may be used to form objects used for biocompatible structures. These objects may be formed using three-dimensional (3D) printing techniques. Cells may be attached for practical applications such as synthetic organs.

### SUMMARY

Embodiments of this disclosure relate to bioinks and bioink compositions. These bioinks may be 3D printed into a hydrogel. The printed hydrogel may support primary cell and induced pluripotent stem cell attachment, proliferation, and spreading. Compounds in the bioink may be modified to incorporate chemical functionality, such as by chemical synthesis means. Incorporating chemical functionality may allow the incorporation of modified material as a component in the bioink. The modifications may allow chemical conjugation of a desired component. The desired component may maintain its cell interactive feature to aid in cell attachment and proliferation. Such incorporation may allow modulation of the bioprinted object's mechanical properties without interfering with cell adhesion.

Disclosed herein are objects formed, e.g., by casting, flood curing, photocuring, photopylmerization, layer by layer printing or extruding objects using the materials disclosed herein. The objects may be organ tissue replacements. The objects may be other objects of commercial value. Embodiments of this disclosure relate to 3D printed objects and methods of forming therein.

Embodiments of this disclosure relate to systems and methods of modifying extracellular matrices (ECMs) to improve cell attachment and interaction with the resulting framework. Disclosed herein is the resulting framework and methods of cell attachment and interaction as well as objects produced therein. As shown herein, extracellular matrices (ECMs) such as collagen I, gelatin, elastin, and fibronectin may be functionalized with groups such as methacrylate groups to enable incorporation into photo-crosslinkable hydrogels. Incorporation of extracellular matrices may enable cell attachment and interaction within hydrogels, rendering the hydrogel biocompatible.

Embodiments of this disclosure relate to a composition comprising a cross-linked (meth)acrylated extracellular matrices (ECM) material and a non-(meth)acryalated ECM material. The composition may have a ratio of (meth)acrylated ECM material to non-(meth)acrylated ECM material of about 5:1 to about 1:5 (e.g. about 5:1, about 4:1, about 3:1, about 2:1, about 1:1, about 1:2, about 1:3, about 1:4, or about 1:5). The (meth)acrylated ECM material may have a degree of (meth)acrylation of about 5 to about 95 percent (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%). The ECM material may be selected from collagen, gelatin, elastin, and fibronectin. The ECM material may be collagen I. The (meth)acrylated ECM material may include a mono- or di-(meth)acrylated ECM or ECM-like material.

The ECM or ECM-like material may be selected from RGD, KQAGDV (SEQ ID NO: 1), YIGSR (SEQ ID NO: 2), REDV (SEQ ID NO: 3), IKVAV (SEQ ID NO: 4), RNIAEIIKDI (SEQ ID NO: 5), KHIFSDDSSE (SEQ ID NO: 6), VPGIG (SEQ ID NO: 7), FHRRIKA (SEQ ID NO: 8), KRSR (SEQ ID NO: 9), APGL (SEQ ID NO: 10), VRN, AAAAAAAAA (SEQ ID NO: 11), GGLGPAGGK (SEQ ID NO: 12), GVPGI (SEQ ID NO: 13), LPETG(G)n (SEQ ID NO: 14), and IEGR (SEQ ID NO: 15). The ECM or ECM-like material may be a sequence sensitive to a protease. The protease may be selected from Arg-C proteinase, Asp-N endopeptidase, BNPS-Skatole, Caspase 1-10, Chymotrypsin-high specificity (C-term to [FYW], not before P), Chymotrypsin-low specificity (C-term to [FYWML (SEQ ID NO: 16)], not before P), Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin, Proline-endopeptidase, Proteinase K, Staphylococcal peptidase I, Thermolysin, Thrombin and Trypsin.

The composition may further comprise a polymeric material. The polymeric material may be hydrophilic. The polymeric material may include one or more of acrylamide, poly(N-isopropyl acrylamide), 2-hydroxylethyl methacrylate, poly (2-hydroxyehtyle methacrylate), triethylene glycol dimethacrylate, tetra (ethylene glycol) dimethacrylte, N, N′-methylene biacrylamide, or amine end-functionalized 4-arm poly(ethylene glycol). The polymeric material may be a polymerized poly(ethylene glycol) di-(meth)acrylate, poly(hydroxy ethyl) (methacrylate), Poly N-hydroxylacrylamide 3-hydroxypropyl acrylate, or Hydroxy butyl acrylate. The polymeric material may be a poly(ethylene glycol) di-(meth)acrylate monomer with a weight average molecular weight (M_{w}) of about 400 to about 20,000. The polymeric material may be a poly(ethylene glycol) di-(meth)acrylate monomer with a M_{w} of about 2000 to about 4000. The polymeric material may be a mixture of any of the aforementioned compositions. The polymeric material may be present in an amount of about 5 to about 50 wt. % of the composition (e.g., about 5 wt. %, about 10 wt. %, about 15 wt. %, about 20 wt. %, about 25 wt. %, about 30 wt. %, about 35 wt. %, about 40 wt. %, about 45 wt. %, or about 50 wt. %). In some embodiments, the polymeric material may be a polymerized poly(ethylene glycol) di-(meth)acrylate monomer that is present in an amount of about 5 to about 50 wt. % of the composition (e.g., about 5 wt. %, about 10 wt. %, about 15 wt. %, about 20 wt. %, about 25 wt. %, about 30 wt. %, about 35 wt. %, about 40 wt. %, about 45 wt. %, or about 50 wt. %).

The composition may support primary cell and/or induced pluripotent stem cell attachment, proliferation, and spreading. The composition may be a molded or 3D printed hydrogel article. The composition may be a molded or 3D hydrogel printed article that has been photo-crosslinked. The molded or 3D printed hydrogel article may be a three-dimensional article of an organ or portion of an organ. The organ may be a mammalian organ, such as an adult human.

Embodiments of this disclosure may relate to a method of manufacturing a three-dimensional article which includes depositing a layer of a printable composition to a surface to obtain a deposited layer, irradiating the deposited layer, and repeating the depositing and irradiating steps until the deposited layers form the three-dimensional article. The printable composition may include, e.g., a (meth)acrylated extracellular matrices (ECM) material, a non-(meth)acrylated ECM material, or a mixture of (meth)acrylated ECM and non-(meth)acrylated ECM, and also a photo initiator. The ECM material may include one or more of collagen, gelatin, elastin, and fibronectin.

The printable composition may also include a poly(ethylene glycol) di-(meth)acrylate monomer. The printable composition may include a mono- or di-(meth)acrylated ECM or ECM-like material. The ECM or ECM-like material may include RGD, PHSRN(GGGERCG)GGRGDSPY (SEQ ID NO: 17 where “GGGERCG” is disclosed as SEQ ID NO: 25), GCREKKRKRLQVQLSIRT (SEQ ID NO: 18), GCREKKTLQPVYEYMVGV (SEQ ID NO: 19), GCREISAFLGIPFAEPPMGPRRFLPPEPKKP (SEQ ID NO: 20), GCRDGPQGWGQDRCG (SEQ ID NO: 21), GCRDVPMSMRGGDRCG (SEQ ID NO: 22), GFOGER (SEQ ID NO: 23), KQAGDV (SEQ ID NO: 1), YIGSR (SEQ ID NO: 2), REDV (SEQ ID NO: 3), IKVAV (SEQ ID NO: 4), RNIAEIIKDI (SEQ ID NO: 5), KHIFSDDSSE (SEQ ID NO: 6), VPGIG (SEQ ID NO: 7), FHRRIKA (SEQ ID NO: 8), KRSR (SEQ ID NO: 9), APGL (SEQ ID NO: 10), VRN, AAAAAAAAA (SEQ ID NO: 11), GGLGPAGGK (SEQ ID NO: 12), GVPGI (SEQ ID NO: 13), LPETG(G)n (SEQ ID NO: 14), and IEGR (SEQ ID NO: 15). The ECM or ECM-like material may include a sequence sensitive to a protease. In some embodiments, the protease may be selected from Arg-C proteinase, Asp-N endopeptidase, BNPS-Skatole, Caspase 1-10, Chymotrypsin-high specificity (C-term to [FYW], not before P), Chymotrypsin-low specificity (C-term to [FYWML (SEQ ID NO: 16)], not before P), Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin, Proline-endopeptidase, Proteinase K, Staphylococcal peptidase I, Thermolysin, Thrombin and Trypsin.

The ratio of (meth)acrylated ECM material to non-(meth)acrylated ECM material may be about 5:1 to about 1:5 (e.g. about 5:1, about 4:1, about 3:1, about 2:1, about 1:1, about 1:2, about 1:3, about 1:4, or about 1:5). The (meth)acrylated ECM material may have a degree of (meth)acrylation of about 5 to about 95 percent (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%). The photo initiator may include lithium phenyl-2,4,6-trimethylbenzoylphosphinate (LAP), Sodium phenyl-2,4,6-trimethylbenzoylphosphinate (NAP)Trimethylbenzoyl based photoinitiators, diphenyl(2,4,6-trimethylbenzoyl)phosphine oxide (TPO nanoparticle) Irgacure class of photoinitiators, ruthenium, and riboflavin, or mixtures thereof.

Embodiments of this disclosure may include a printable composition where one or more additives include polymers, photoactive dye, natural extracellular matrices, photoinitiators, Peptides, amino acids, growth factors, denature extracellular matrices, extracellular matrix fragments or mixtures thereof. The photoactive dye may be a UV dye with absorbance spectra between 300 nm to 420 nm. The photoactive dye may have a wavelength range of 300 nm to 400 nm. The photoactive dye may be non-cytotoxic. The photoactive dye may include a benzyne ring in the molecular structure. The photoactive dye may be quinolone yellow, a UV dye, or a dye with a molecular structure similar thereto. The photoactive dye may be UV 386A dye.

Embodiments also include a printed scaffold. The printed scaffold may be non-cytotoxic when leaching out monomers to the buffer that the scaffolds are placed in. Embodiments may include the composition used to print the aforementioned scaffold. Embodiments may include a formed three-dimensional article. The three-dimensional article may replicate an organ or a portion of an organ.

Embodiments also include the printable composition including a protic solvent. The protic solvent may include water, polyethylene glycol, glycol diacrylate derivatives or mixtures thereof.

Embodiments of this disclosure also relate to the use of functionalized and unfunctionalized extracellular matrices, matrices fragments, peptides and bioactive components formed by the methods disclosed.

### BRIEF DESCRIPTION OF THE DRAWINGS

FIG. 1 shows a schematic of a reaction between collagen with methacrylate anhydride to form methacrylated collagen.

FIG. 2A shows an image and FIG. 2B shows a graph of cell spread, density, and % cell coverage of lung fibroblasts cultured for 7 days on different surfaces: (i) glass (control), (ii) bioink containing 50% DOF collagen, (iii) bioink containing 90% DOF, and (iv) bioink containing 0% DOF collagen and 90% DOF collagen (1:2 ratio). Additional description is provided in Example 1.

FIG. 3A shows an image and FIG. 3B shows a graph of cell spread, density, and % cell coverage of pulmonary artery endothelial cells cultured for 1 day on different surfaces: (i) glass (control), (ii) 9% PEGDA Hybrid and 9% PEGDA. The asterisk (*) indicates a p value of less than 0.05. Additional description is provided in Example 2.

FIG. 4 shows a culture of lung smooth muscle cells attaching, proliferating, and spreading on a 3D printed disk made up of PEGDA MW 3400, 95% DOF collagen and 0% DOF collagen (2:1 ratio) over the course of seven days. Additional description is provided in Example 3.

FIGS. 5A-5C show cells adhesion on different 3D printed objects made using different bioinks and with different ratios of functionalized and non-functionalized collagen. Additional description is provided in Example 4.

FIG. 6 shows lung fibroblast cell attachment and proliferation on a 3D printed objects made with bioink containing functionalized and non-functionalized collagen supports over a seven day period. Additional description is provided in Example 5.

FIGS. 7A-7B show the effect of HEAA content on cell adhesion properties. Additional description is provided in Example 6.

FIGS. 8A-8E show the effects of CollMA DOF on cell adhesion and proliferation (>90%, ˜50%, hybrid (50% Non-MA, 50% HM)). Additional description is provided in Example 7.

FIG. 9 shows a comparison of cell density, cell spread, and cell coverage of PAEC cells. Additional description is provided in Example 8.

FIGS. 10A-10D shows the differences in cells spread, cell density, and cell coverage on a 5% PEGDA 3D-printed substrate with different degrees of functionalization. Additional description is provided in Example 10.

FIGS. 11A-11D show the cells coverage, cell spread, and cell density characteristics of [insert] cells on different 3D printed disks: (FIG. 11A) Image of 3D printed disks on platform; (FIG. 11B) Day 1 Analysis of Percent Area Coverage, Cell Spread, and Cell density of AC42 1 mm disks, AC42 3 mm disks, Leaching controls and Glass controls; (FIG. 11C) Day 4 Analysis of Percent Area Coverage, Cell Spread, and Cell density of AC42 1 mm disks, AC42 3 mm disks, Leaching controls and Glass controls; (FIG. 11D) Day 7 Analysis of Percent Area Coverage, Cell Spread, and Cell density of AC42 1 mm disks, AC42 3 mm disks, Leaching controls and Glass controls.

FIGS. 12A-12B show the cells coverage, cell spread, and cell density characteristics of cells on different 3D printed disks: (A) Comparison of Percent Area Coverage, Cell Spread, and Cell Density Comparison of AC42 1 mm disks, 3 mm disks, Leaching Controls, and Glass Controls seeded with LFN, PAEC or SAEC seeds; (B) Comparison of Percent Area Coverage, Cell Spread, and Cell Density Comparison of AC42 1 mm disks and 3 mm disks seeded with LFN, PAEC or SAEC seeds.

FIG. 13 shows certain embodiments of biologically active peptides that can be incorporated into bioinks of the present disclosure. Figure discloses SEQ ID NOS 26, 27 (where “GGGERCG” is disclosed as SEQ ID NO: 25), 18, 28, 19, 20, 22, 29, and 30, respectively, in order of appearance.

FIGS. 14A-14C show the cells coverage, cell spread, and cell density characteristics of cells on different 3D printed disks. Additional details are in Example 13.

FIGS. 15A-15B show embodiments where peptides enhance bioactivity in bioinks to promote cell attachment. Additional details are disclosed in Example 14.

FIGS. 16A-16B show embodiments where peptides enhance bioactivity in bioinks to promote cell attachment. Additional details are disclosed in Example 14.

FIGS. 17A-17B show embodiments where peptides enhance bioactivity in bioinks to promote cell attachment. Additional details are disclosed in Example 14.

### DETAILED DESCRIPTION

As used herein, “3D printing” refers to any technique used to make a three-dimensional object using a digital model of that object. Exemplary 3D printing techniques include selective laser sintering (SLS) method, a fused deposition modeling (FDM) method, a 3D inkjet printing method, a digital light processing (DLP) method, and a stereolithography method.

As used herein, “printable ink” and “printable composition” refer to any composition that can be used to form an object using a 3D printing technique. A “bioink” is a printable ink that forms a material with one or more desired biocompatibility properties. For example, a bioink may contain one or more materials that facilitate adhesion and proliferation of desired cell types. The printed object may support primary cell and induced pluripotent stem cell attachment, proliferation, and spreading. In some cases, the bioink can be formed into a hydrogel. Compounds in the bioink may be selected or modified to incorporate chemical functionality, such as by chemical synthesis means. Chemical functionality may allow the incorporation of modified material as a component in the bioink. The modifications may allow chemical conjugation of a desired component. The desired component may maintain its cell interactive feature. Such incorporation may allow modulation of the printed object's mechanical properties without interfering with cell adhesion.

As used herein, “extracellular matrix” and “ECM” refer to natural and synthetic ECMs as well as one or more materials that constitute an ECM. For example, ECM can refer to a naturally-occurring ECM or an ECM made using synthetic techniques. ECM can also refer to one or more materials that constitute a naturally-occurring ECM, such as collagen (natural or synthetic). In some cases, “ECM material” will be used to refer to specific materials. ECM can be made using various techniques, including 3D printing. The ECMs can be made using a hydrogel material.

As used herein, “extracellular matrix” and “ECM” refer to natural and synthetic ECMs as well as one or more materials that constitute an ECM. For example, ECM can refer to a naturally-occurring ECM or an ECM made using synthetic techniques. ECM can also refer to one or more materials that constitute a naturally-occurring ECM, such as collagen either natural or synthetic. In some cases, “ECM material” will be used to refer to specific materials. ECM can be made using various techniques, including 3D printing. The ECMs can be made using a hydrogel material. ECM matrix material, such as collagen I, gelatin, elastin, and fibronectin, may be functionalized with methacrylate groups to enable incorporation into photo-crosslinkable hydrogels. Incorporation of ECM materials to other materials and objects, such as 3D printed materials, may increase biocompatibility and enable cell attachment and interaction within the materials and objects. The extent to which a material enables cell attachment can vary based on the amount of ECM material, the availability of binding sites on or within the material, the surface charge of the material, the polarity of the material, as well as the mechanical properties of the material.

The present application incorporates by reference in their entirety each of the following documents: (a) U.S. provisional application No. 63/185,300 filed May 6, 2021 titled “CONTROLLING THE SIZE OF 3D PRINTING HYDROGEL OBJECTS USING HYDROPHILIC MONOMERS, HYDROPHOBIC MONOMERS, AND CROSSLINKERS” and U.S. non-provisional and/or PCT application(s) under the same title filed on May 6, 2022; (b) U.S. provisional application No. 63/185,302 filed May 6, 2021 titled “MODIFIED 3D-PRINTED OBJECTS AND THEIR USES” and U.S. non-provisional and/or PCT application(s) under the same title filed on May 6, 2022; (c) U.S. provisional application No. 63/185,305 filed May 6, 2021 titled “PHOTOCURABLE REINFORCEMENT OF 3D PRINTED HYDROGEL OBJECTS” and U.S. non-provisional and/or PCT application(s) under the same title filed on May 6, 2022; (d) U.S. provisional application No. 63/185,299 filed May 6, 2021 titled “ADDITIVE MANUFACTURING OF HYDROGEL TUBES FOR BIOMEDICAL APPLICATIONS” and U.S. non-provisional and/or PCT application(s) under the same title filed on May 6, 2022; (e) U.S. provisional application No. 63/185,298 filed May 6, 2021 titled “MICROPHYSIOLOGICAL 3-D PRINTING AND ITS APPLICATIONS” and U.S. non-provisional and/or PCT application(s) under the same title filed on May 6, 2022.

ECMs may be functionalized with methacrylate groups by substituting the lysine residue on the amine group with, e.g., methacrylate anhydride (MAA). The degree of (meth)acrylation of an ECM can be defined by the percentage of available amine groups which have been modified with AA or MAA. A higher degree of (meth)acrylation correlates with more AA or MAA modified amine groups resulting in less free amine groups.

The degree of functionalization may be varied to achieve a particular degree or type of functionalization. A hybrid form of functionalized and unfunctionalized components may be used. In some embodiments, collagen may be modified to form a (meth)acrylated collagen. A hybrid combination may be used, which in this instance could refer to a hydrogel containing both methacrylated and non-methacrylated collagen. 3D printed objects made of poly(ethylene glycol) diacrylate containing (meth)acrylated and non-(meth)acryalted collagen may support attachment, spreading, and/or proliferation of, e.g., lung-derived cells, including fibroblasts, endothelial cells, and smooth muscles.

A variety of biomaterials may be used in the place of collagen or in addition to collagen. These biomaterials may include collagen IV, fibronectin, gelatin, collagen type III, short peptides (such as RGD), fragments of ECM proteins, proteoglycans, glycosamoinoglycans, hyaluronic acid or any other ECM from any species. The available binding sites for these ECMs can be altered via functionalization or modification by different chemical groups. These chemical groups may bind to amine groups or other groups which cells have affinity to.

The ECM compositions may be formulated for use in casting, extrusion, or 3D printing applications, such as use of bioinks to form the compositions. In some embodiments, these compositions increase cell adhesion, proliferation, spreading and migration into 3D printed materials, including hydrogels. These 3D printed materials may be used for biological, biomedical, medical device, and/or healthcare applications. These printed materials may be used for diagnostic devices. These printed hydrogels may be useful for applications that need surfaces that which require tight control of binding sites to the surface of biological components.

Certain embodiments of this disclosure relate to a composition comprising a cross-linked (meth)acrylated and non-(meth)acrylated ECM material. The composition may have a ratio of (meth)acrylated ECM material to non-(meth)acrylated ECM material of about 5:1 to about 1:5 (e.g. about 5:1, about 4:1, about 3:1, about 2:1, about 1:1, about 1:2, about 1:3, about 1:4, or about 1:5). The (meth)acrylated ECM material may have a degree of (meth)acrylation of about 5 to about 95 percent (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%). The ECM material may be selected from collagen, gelatin, elastin, and fibronectin. The ECM material may be collagen I. The (meth)acrylated ECM material may include a mono- or di-(meth)acrylated ECM or ECM-like material.

The ECM may comprise one or more peptides. Non-limiting examples of suitable peptides include RGD, PHSRN(GGGERCG)GGRGDSPY (SEQ ID NO: 17 where “GGGERCG” is disclosed as SEQ ID NO: 25), GCREKKRKRLQVQLSIRT (SEQ ID NO: 18), GCREKKTLQPVYEYMVGV (SEQ ID NO: 19), GCREISAFLGIPFAEPPMGPRRFLPPEPKKP (SEQ ID NO: 20), GCRDGPQGWGQDRCG (SEQ ID NO: 21), GCRDVPMSMRGGDRCG (SEQ ID NO: 22), GFOGER (SEQ ID NO: 23), KQAGDV (SEQ ID NO: 1), YIGSR (SEQ ID NO: 2), REDV (SEQ ID NO: 3), IKVAV (SEQ ID NO: 4), RNIAEIIKDI (SEQ ID NO: 5), KHIFSDDSSE (SEQ ID NO: 6), VPGIG (SEQ ID NO: 7), FHRRIKA (SEQ ID NO: 8), KRSR (SEQ ID NO: 9), APGL (SEQ ID NO: 10), VRN, AAAAAAAAA (SEQ ID NO: 11), GGLGPAGGK (SEQ ID NO: 12), GVPGI (SEQ ID NO: 13), LPETG(G)n (SEQ ID NO: 14), and IEGR (SEQ ID NO: 15). Suitable peptides also include peptide materials that mimic features of native ECMs, including integrin binding, syndecan binding, ECM deposition, and/or MMP-dependent remodeling. The peptide may be present in a printable composition in an amount of about 0.5 mM to about 5 mM (e.g., about 0.5 mM, about 1 mM, about 1.5 mM, about 2 mM, about 2.5 mM, about 3 mM, about 3.5 mM, about 4 mM, about 4.5 mM, or about 5 mM). In some cases, the ECM material can contain from about 0.5 mM of peptide to about 10 mM of peptide. In other cases, the ECM material can contain from about 5 mM of peptide to about 20 mM of peptide. In other cases, the ECM material can contain from about 10 mM of peptide to about 100 mM of peptide. The ECM or ECM-like material may be an amino acid sequence sensitive to a protease. The protease may be selected from Arg-C proteinase, Asp-N endopeptidase, BNPS-Skatole, Caspase 1-10, Chymotrypsin-high specificity (C-term to [FYW], not before P), Chymotrypsin-low specificity (C-term to [FYWML (SEQ ID NO: 16)], not before P), Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin, Proline-endopeptidase, Proteinase K, Staphylococcal peptidase I, Thermolysin, Thrombin and Trypsin.

The composition may further comprise a polymeric material. The polymeric material may be hydrophilic. The polymeric material may be acrylamide, poly(N-isopropyl acrylamide), 2-hydroxylethyl methacrylate, poly (2-hydroxyethyl methacrylate), triethylene glycol dimethacrylate, tetra (ethylene glycol) dimethacryalte, N, N′-methylene bisacrylamide, or amine end-functionalized 4-arm poly(ethylene glycol). The polymeric material may be a polymerized poly(ethylene glycol) di-(meth)acrylate, poly(hydroxy ethyl) (methacrylate), Poly N-hydroxyacrylamide 3-hydroxypropyl acrylate, or Hydroxy butyl acrylate. The polymeric material may be a poly(ethylene glycol) di-(meth)acrylate monomer with a weight average molecular weight (M_{w}) of about 400 to about 20,000. The polymeric material may be a poly(ethylene glycol) di-(meth)acrylate monomer with a M_{w} of about 2000 to about 4000. The polymeric material may be a mixture of any of the aforementioned compositions. The polymeric material may be present in an amount of about 5 to 50 wt. % of the composition (e.g., about 5 wt. %, about 10 wt. %, about 15 wt. %, about 20 wt. %, about 25 wt. %, about 30 wt. %, about 35 wt. %, about 40 wt. %, about 45 wt. %, or about 50 wt. %). In some embodiments, the polymeric material may be a polymerized poly(ethylene glycol) di-(meth)acrylate monomer that is present in an amount of about 5 to about 50 wt. % of the composition (e.g., about 5 wt. %, about 10 wt. %, about 15 wt. %, about 20 wt. %, about 25 wt. %, about 30 wt. %, about 35 wt. %, about 40 wt. %, about 45 wt. %, or about 50 wt. %).

Printable Compositions

Embodiments of this disclosure may relate to a method of manufacturing a three-dimensional article which includes depositing a layer of a printable composition to a surface to obtain a deposited layer, irradiating the deposited layer, and repeating the depositing and irradiating steps until the deposited layers form the three-dimensional article. The printable composition may include a (meth)acrylated extracellular matrices (ECM) material, a non-(meth)acrylated ECM material, and a photo initiator. The ECM material may include collagen, gelatin, elastin, and fibronectin.

The printable composition may include a poly(ethylene glycol) di-(meth)acrylate monomer. The printable composition may include a mono- or di-(meth)acrylated ECM or ECM-like material. The ECM or ECM-like material may comprise one or more of RGD, PHSRN(GGGERCG)GGRGDSPY (SEQ ID NO: 17 where “GGGERCG” is disclosed as SEQ ID NO: 25), GCREKKRKRLQVQLSIRT (SEQ ID NO: 18), GCREKKTLQPVYEYMVGV (SEQ ID NO: 19), GCREISAFLGIPFAEPPMGPRRFLPPEPKKP (SEQ ID NO: 20), GCRDGPQGWGQDRCG (SEQ ID NO: 21), GCRDVPMSMRGGDRCG (SEQ ID NO: 22), GFOGER (SEQ ID NO: 23), KQAGDV (SEQ ID NO: 1), YIGSR (SEQ ID NO: 2), REDV (SEQ ID NO: 3), IKVAV (SEQ ID NO: 4), RNIAEIIKDI (SEQ ID NO: 5), KHIFSDDSSE (SEQ ID NO: 6), VPGIG (SEQ ID NO: 7), FHRRIKA (SEQ ID NO: 8), KRSR (SEQ ID NO: 9), APGL (SEQ ID NO: 10), VRN, AAAAAAAAA (SEQ ID NO: 11), GGLGPAGGK (SEQ ID NO: 12), GVPGI (SEQ ID NO: 13), LPETG(G)n (SEQ ID NO: 14), and IEGR (SEQ ID NO: 15). Suitable peptides also include peptide materials that mimic features of native ECMs, including integrin binding, syndecan binding, ECM deposition, and/or MMP-dependent remodeling. The peptide may be present in a printable composition in an amount of about 0.5 mM to about 5 mM (e.g., about 0.5 mM, about 1 mM, about 1.5 mM, about 2 mM, about 2.5 mM, about 3 mM, about 3.5 mM, about 4 mM, about 4.5 mM, or about 5 mM). The ECM or ECM-like material may include a sequence sensitive to a protease. The protease may be selected from Arg-C proteinase, Asp-N endopeptidase, BNPS-Skatole, Caspase 1-10, Chymotrypsin-high specificity (C-term to [FYW], not before P), Chymotrypsin-low specificity (C-term to [FYWML (SEQ ID NO: 16)], not before P), Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin, Proline-endopeptidase, Proteinase K, Staphylococcal peptidase I, Thermolysin, Thrombin and Trypsin.

The ratio of (meth)acrylated ECM material to non-(meth)acrylated ECM material may be about 5:1 to about 1:5 (e.g. about 5:1, about 4:1, about 3:1, about 2:1, about 1:1, about 1:2, about 1:3, about 1:4, or about 1:5). The (meth)acrylated ECM material may have a degree of (meth)acrylation of about 5 to about 95 percent (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%). The photo initiator may include lithium phenyl-2,4,6-trimethylbenzoylphosphinate (LAP), Sodium phenyl-2,4,6-trimethylbenzoylphosphinate (NAP)Trimethylbenzoyl based photoinitiators, diphenyl(2,4,6-trimethylbenzoyl)phosphine oxide (TPO nanoparticle) Irgacure class of photoinitiators, ruthenium, and riboflavin, or mixtures thereof.

Embodiments of this disclosure may include a printable composition where one or more additives include polymers, photoactive dye, natural extracellular matrices, photoinitiators, Peptides, amino acids, growth factors, denature extracellular matrices, extracellular matrix fragments or mixtures thereof. The photoactive dye may be a UV dye with absorbance spectra between 300 nm to 420 nm. The photoactive dye may have a wavelength range of 300 nm to 400 nm. The photoactive dye may be non-cytotoxic. The photoactive dye may include a benzyne ring in the molecular structure. The photoactive dye may be quinolone yellow, a UV dye, or a dye with a molecular structure similar thereto. The photoactive dye may be UV 386A dye.

Printable compositions described herein can be used to make scaffolds using 3D printing. The printed scaffold may be formulated, such as be using appropriately-formulated bioinks, to be non-cytotoxic when placed in suitable buffers. The bioink selected for forming the scaffold can be selected based on the desired biocompatibility of the resulting scaffold. For example, the scaffold may support the adhesion, growth, and proliferation of selected cell types, such as lung-derived cells.

Printable compositions may comprise a protic solvent. The protic solvent can comprise water, polyethylene glycol, glycol diacrylate derivatives or mixtures thereof. In some cases, the printable composition can contain a buffer solution of HEPES (4-(2-hydroxyethyl)-1-piperazineethanesulfonic acid) or PBS (Phosphate buffered saline). The polyethylene glycol diacrylates can vary from around 1 wt. % to around 20 wt. %. Non-limiting examples of the polyethylene glycol diacrylates include oxyethylene diacrylate 3400 (PEGDA3400), polyethylene diacrylate 6000 (PEGDA6000), polyethylene diacrylate 575 (PEGDA575), and mixtures thereof. The printable composition can contain from about 1 wt. % to about 20 wt. % of HPA or HBA. The printable material can also contain about 0.5 wt. % to about 3 wt. % N-Hydroxyethyl Acrylamide (HEAR). The printable material can also contain about 0.5 wt. % to about 10 wt. % the (meth)acrylated ECM material and/or non-(meth)acrylated ECM material. Further, the printable material can also contain about 0.5 wt. % to about 3 wt. % of PEG-AcrylateCGRGDS, such as PEG₃₄₀₀AcrylateCGRGDS. The printable material can contain a photoinitiator from about 0.5% to about 5% and a photoabsorbent dye of about 0.1% to about 5%.

The composition may support primary cell and/or induced pluripotent stem cell attachment, proliferation, and spreading. The composition may be a molded or 3D printed hydrogel article. The composition may be a molded or 3D hydrogel printed article that has been photo-crosslinked. The molded or 3D printed hydrogel article may be a three-dimensional article of an organ. The organ may be a mammalian organ.

The printable compositions described herein can be formed into three-dimensional objects that mimic or replicate an organ or a portion of an organ. For example, the printable compositions described herein can be formed into a structure that mimics or replicates the architecture of the lung, such as by using 3D printing techniques. The printable compositions can be used to form a scaffold for adhesion and growth of cells resulting in a structure that has one or more desired properties of an organ, such as a structure that can be perform the gas exchange functions of a lung. These objects can comprise a hydrogel. The organ or portion of an organ can be a human lung in a preferred embodiment. The shape of the 3D object is not particularly limited, and may be in a shape of a tube, or substantially the same shape, size, and/or has the same relative dimensions of an organ or a fragment of an organ.

In some embodiments, the object formed from the printable composition is substantially the same shape, size, and/or has the same relative dimensions of an organ or a fragment of an organ. In certain embodiments, the organ or fragment of the organ comprises a vessel, trachea, bronchi, esophagus, ureter, renal tubule, bile duct, renal duct, bile duct, hepatic duct, nerve conduit, CSF shunt, lung, kidney, heart, liver, spleen, brain, gallbladder, stomach, pancreas, bladder, lymph vessel, skeletal bone, cartilage, skin, intestine, a muscle, larynx, or pharynx. In additional embodiments, the vessel shape comprises a pulmonary artery, renal artery, coronary artery, peripheral artery, pulmonary vein, or renal vein. In certain embodiments, the structure comprises a hemodialysis graft. Other embodiments include where the structure is substantially is the shape of a lung lobe, lung, airway tree of a lung, lung vasculature, or a combination thereof. In some embodiments, the reinforcement comprises maintaining air-flow or blood (or fluid) flow through the structure when an external pressure is applied to the structure.

Embodiments of this disclosure relate to the use of functionalized and nonfunctionalized extracellular matrices, matrices fragments, peptides and bioactive components formed by the methods disclosed.

3D Compositions

Embodiments of this disclosure may relate to a method of manufacturing a three-dimensional article which includes depositing a layer of a printable composition to a surface to obtain a deposited layer, irradiating the deposited layer, and repeating the depositing and irradiating steps until the deposited layers form the three-dimensional article. The printable composition may include a hydrogel material, modified or unmodified extracellular matrices (ECM) material and a photo initiator.

The three-dimensional (3D) hydrogel structure is not particularly limited, and can be, e.g., a composite structure made of one or more different polymerized monomers. Hydrogel materials that may be used in the invention may be known to those having ordinary skill in the art, as are methods of making the same. For example, a hydrogel as described in Caló et al., European Polymer Journal Volume 65, April 2015, Pages 252-267 may be used. In some embodiments, the hydrogel structure comprises a polymerized (meth)acrylate and/or (meth)acrylamide hydrogel. In some embodiments, the structure comprises a polymer comprising polymerized poly(ethylene glycol) di(meth)acrylate, polymerized poly(ethylene glycol) di(meth)acrylamide, polymerized poly(ethylene glycol) (meth)acrylate/(methacrylamide), poly(ethylene glycol)-block-poly(ε-caprolactone), polycaprolactone, polyvinyl alcohol, gelatin, methylcellulose, hydroxyethyl methyl cellulose, hydroxypropyl methyl cellulose, polyethylene oxide, polyacrylamides, polyacrylic acid, polymethacrylic acid, salts of polyacrylic acid, salts of polymethacrylic acid, poly(2-hydroxyethyl methacrylate), polylactic acid, polyglycolic acid, polyvinylalcohol, polyanhydrides such as poly(methacrylic) anhydride, poly(acrylic) anhydride, polysebasic anhydride, collagen, poly(hyaluronic acid), hyaluronic acid-containing polymers and copolymers, polypeptides, dextran, dextran sulfate, chitosan, chitin, agarose gels, fibrin gels, soy-derived hydrogels, alginate-based hydrogels, poly(sodium alginate), hydroxypropyl acrylate (HPA), lithium phenyl-2,4,6-trimethylbenzoylphosphinate (LAP), sodium phenyl-2,4,6-trimethylbenzoylphosphinate (NAP) and combinations thereof. In some embodiments, the M_{w} of the hydrogel polymer is about 400 Da, 500 Da, 600 Da, 700 Da, 800 Da, 900 Da, 1000 Da, 1100 Da, 1200 Da, 1300 Da, 1400 Da, 1500 Da, 1600 Da, 1700 Da, 1800 Da, 1900 Da, 2000 Da, 2100 Da, 2200 Da, 2300 Da, 2400 Da, 2500 Da, 2600 Da, 2700 Da, 2800 Da, 2900 Da, 3000 Da, 3100 Da, 3200 Da, 3300 Da, 3400 Da, 3500 Da, 3600 Da, 3700 Da, 3800 Da, 3900 Da, 4000 Da, 4100 Da, 4200 Da, 4300 Da, 4400 Da, 4500 Da, 4600 Da, 4700 Da, 4800 Da, 4900 Da, 5000 Da, 5100 Da, 5200 Da, 5300 Da, 5400 Da, 5500 Da, 5600 Da, 5700 Da, 5800 Da, 5900 Da, 6000 Da, 6100 Da, 6200 Da, 6300 Da, 6400 Da, 6500 Da, 7000 Da, 7500 Da, 8000 Da, 8500 Da, 9000 Da, 9500 Da, 10000 Da, 15000 Da, or 20000 Da. In some embodiments, the hydrogel polymer comprises NAP as a primary ingredient, and further comprising one or more peptide and/or collagen described herein that is functionalized with PEGDAs.

In some embodiments, the hydrogel comprises a cross-linked polymer. In some embodiments, the polymer is about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100% cross-linked, based on the percentage of the cross-linkable moieties in the polymer. Cross-linkable moieties may include, for example, (meth)acrylate groups.

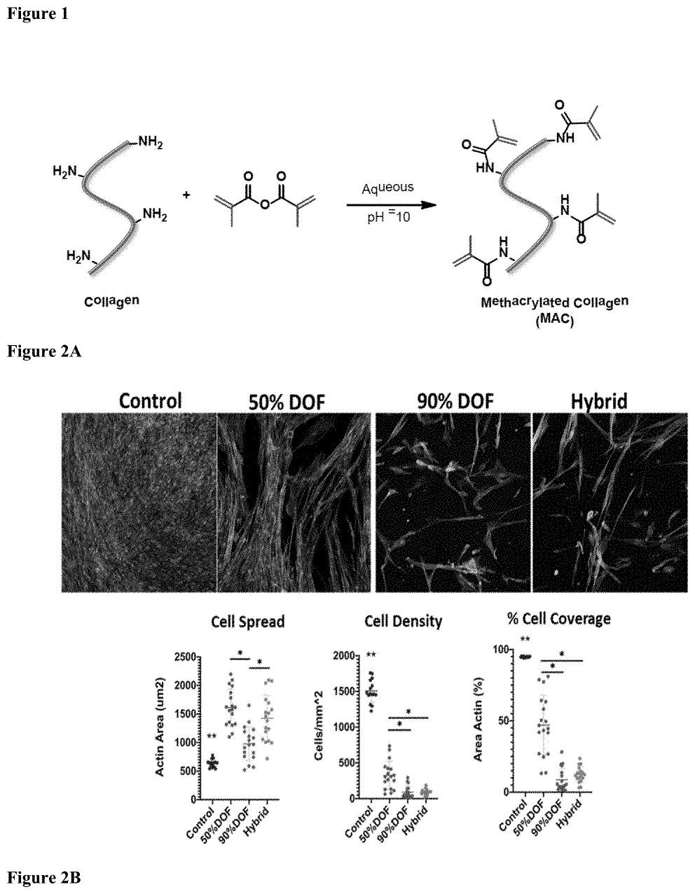

Embodiments of this disclosure relate to the use of functionalized and nonfunctionalized extracellular matrices, matrices fragments, peptides and bioactive components formed by the methods disclosed. In some cases, these extracellular matrices may be formed from materials including collagen, gelatin, elastin, and/or fibronectin. In some cases this compound, such as collagen, may be reacted with methacrylate anhydride to form methacrylated compound, such as methacrylated collagen as shown in FIG. 1.

As disclosed herein, the degree of functionalized (DOF) collagen in hydrogels may be varied, for instance, in the ratios of (meth)acrylated collagen and non-(meth)acrylated collagen. In some cases, the collagen may be hybrid, or contain both functionalized and nonfunctionalized components. For instance, hybrid may refer to a hydrogel containing both (meth)acrylated and non-(meth)acrylated collagen. 3D printed objects made of Poly(ethylene glycol) diacrylate containing (meth)acrylated and non (meth)acrylated collagen may support attachment, spreading, and/or proliferation of lung derived fibroblasts, endothelial cells, and smooth muscles.

As disclosed herein, alternate biomaterials may be used in the place of collagen. These biomaterials may include collagen IV, fibronectin, gelatin, collagen type III, short peptides (such as RGD), fragments of ECM proteins, proteoglycans, glycosaminoglycans, hyaluronic acid or any other extracellular matrix from any species.

The printable composition may be modified to enhance cell attachment and/or mechanical properties. These printable compositions or inks may have unique mechanical properties or reactivity orthogonal to acrylate reactivity. Components of the inks or printable composition may include a poly(ethylene glycol) di-(meth)acrylate monomer. The printable composition may include a mono- or di-(meth)acrylated ECM or ECM-like material. The ECM or ECM-like material may include one or more of RGD, PHSRN(GGGERCG)GGRGDSPY (SEQ ID NO: 17 where “GGGERCG” is disclosed as SEQ ID NO: 25), GCREKKRKRLQVQLSIRT (SEQ ID NO: 18), GCREKKTLQPVYEYMVGV (SEQ ID NO: 19), GCREISAFLGIPFAEPPMGPRRFLPPEPKKP (SEQ ID NO: 20), GCRDGPQGWGQDRCG (SEQ ID NO: 21), GCRDVPMSMRGGDRCG (SEQ ID NO: 22), GFOGER (SEQ ID NO: 23), KQAGDV (SEQ ID NO: 1), YIGSR (SEQ ID NO: 2), REDV (SEQ ID NO: 3), IKVAV (SEQ ID NO: 4), RNIAEIIKDI (SEQ ID NO: 5), KHIFSDDSSE (SEQ ID NO: 6), VPGIG (SEQ ID NO: 7), FHRRIKA (SEQ ID NO: 8), KRSR (SEQ ID NO: 9), APGL (SEQ ID NO: 10), VRN, AAAAAAAAA (SEQ ID NO: 11), GGLGPAGGK (SEQ ID NO: 12), GVPGI (SEQ ID NO: 13), LPETG(G)n (SEQ ID NO: 14), and IEGR (SEQ ID NO: 15). Suitable peptides also include peptide materials that mimic features of native ECMs, including integrin binding, syndecan binding, ECM deposition, and/or MMP-dependent remodeling. The peptide may be present in a printable composition in an amount of about 0.5 mM to about 5 mM (e.g., about 0.5 mM, about 1 mM, about 1.5 mM, about 2 mM, about 2.5 mM, about 3 mM, about 3.5 mM, about 4 mM, about 4.5 mM, or about 5 mM). Alternatively or additionally, the 3D printed object may be further surface modified with one or more peptide. Surface modification may be achieved by reacting unreacted (meth)acrylate moieties in the 3D printed object with one or more peptide by contacting the unreacted moieties with a solution comprising the one or more peptide. It is to be understood that this disclosure includes surface modification with other ECM or ECM-like materials disclosed herein in the same manner as described above for peptides. The ECM or ECM-like material may include a sequence sensitive to a protease. The protease may be selected from Arg-C proteinase, Asp-N endopeptidase, BNPS-Skatole, Caspase 1-10, Chymotrypsin-high specificity (C-term to [FYW], not before P), Chymotrypsin-low specificity (C-term to [FYWML (SEQ ID NO: 16)], not before P), Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin, Proline-endopeptidase, Proteinase K, Staphylococcal peptidase I, Thermolysin, Thrombin and Trypsin.

The available binding sites for the extracellular matrices can be altered via functionalization or modification by different chemical groups. These chemical groups may bind to amine groups or other groups which cells have affinity to. Below are examples of mono- and di-(meth)acrylates with ECM or bioactive components R₁ that can be used to make ECM/ECM-like printable compositions or inks.

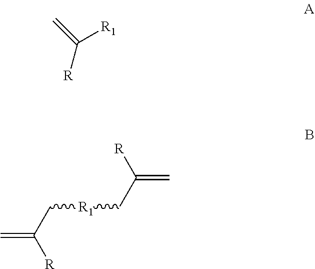

R can be a hydrogen or methyl group. R₁ can be any of the following:

| | |
|---|---|
| RGD | Fibronectin, Vitronectin Cell adhesion |
| PHSRNKRGD | Fibronectin cell adhesion |
| GCREKKRKRLQVQLSIRT (SEQ ID NO: 18) | Laminin cell adhesion |
| GCREKKTLQPVYEYMVGV (SEQ ID NO: 19) | Peptide with affinity for fibronectin |
| GCREISAFLGIPFAEPPMGPRRFLPPEPKKP (SEQ ID NO: 20) | Peptide with affinity for Col |
| IV and LMNGCRDGPQGWGQDRCG (SEQ ID NO: 24) | Cell degradable peptide |
| GCRDVPMSMRGGDRCG (SEQ ID NO: 22) | Cell degradable peptide |
| KQAGDV (SEQ ID NO: 1) | Smooth muscle cell adhesion |
| YIGSR (SEQ ID NO: 2) | Laminin B1 Cell adhesion |
| REDV (SEQ ID NO: 3) | Fibronectin Endothelial cell adhesion |
| IKVAV (SEQ ID NO: 4) | Laminin Neurite extension |
| RNIAEIIKDI (SEQ ID NO: 5) | Laminin B2 Neurite extension |
| KHIFSDDSSE (SEQ ID NO: 6) | Neural cell adhesion molecules Astrocyte adhesion |
| VPGIG (SEQ ID NO: 7) | Elastin Enhance elastic modulus of artificial ECM |
| FHRRIKA (SEQ ID NO: 8) | Heparin binding domain Improve osteoblastic |
| | mineralization |
| KRSR (SEQ ID NO: 9) | Heparin binding domain Osteoblast adhesion |
| APGL (SEQ ID NO: 10) | Collagenase mediated degradation |
| VRN | Plasmin mediated degradation |
| AAAAAAAAA (SEQ ID NO: 11) | Elastase mediated degradation |
| GGLGPAGGK (SEQ ID NO: 12) | Protease sensitive peptide |
| GVPGI (SEQ ID NO: 13) | Elastin related for mechanical stability |
| LPETG(G)ₙ (SEQ ID NO: 14) | Sortase mediated ligation |
| IEGR (SEQ ID NO: 15) | Protease sensitive |

ECM components or protease digests of ECM components
Mono-, oligo-, poly-saccharides (Hyaluronic acid, heparan, aggrecan, chondroitin, sialic acid)

Redox reactive:
Sulfhydryl (—SH)
Disulfide (S—S)

R₁ can also be any of the following sequences sensitive to the proteases below, where [P4 . . . P2′] is commonly accepted nomenclature for protease cleavage site sequence specification:

TABLE 1

| Enzyme name | P4 | P3 | P2 | P1 | P1′ | P2′ |
|---|---|---|---|---|---|---|
| Arg-C proteinase | — | — | — | R | — | — |
| Asp-N endopeptidase | — | — | — | — | D | — |
| BNPS-Skatole | — | — | — | W | — | — |
| Caspase 1 | F, W, Y, or L | — | H, A or T | D | not P, E, D, | — |
| | | | | | Q, K or R | |
| Caspase 2 | D | V | A | D | not P, E, D, | — |
| | | | | | Q, K or R | |
| Caspase 3 | D | M | Q | D | not P, E, D, | — |
| | | | | | Q, K or R | |
| Caspase 4 | L | E | V | D | not P, E, D, | — |
| | | | | | Q, K or R | |
| Caspase 5 | L or W | E | H | D | — | — |
| Caspase 6 | V | E | H or I | D | not P, E, D, | — |
| | | | | | Q, K or R | |
| Caspase 7 | D | E | V | D | not P, E, D, | — |
| | | | | | Q, K or R | |
| Caspase 8 | I or L | E | T | D | not P, E, D, | — |
| | | | | | Q, K or R | |
| Caspase 9 | L | E | H | D | — | — |
| Caspase 10 | I | E | A | D | — | — |
| Chymotrypsin-high | — | — | — | F or Y | not P | — |
| specificity (C-term to | — | — | — | W | not M or P | — |
| [FYW], not before P) | | | | | | |
| Chymotrypsin-low | — | — | — | F, L or Y | not P | — |
| specificity (C-term to | — | — | — | W | not M or P | — |
| [FYWML (SEQ ID | — | — | — | M | not P or Y | — |
| NO: 16)], not before | — | — | — | H | not D, M, P or | — |
| P) | | | | | W | |
| Clostripain | — | — | — | R | — | — |
| (Clostridiopeptidase | | | | | | |
| B) | | | | | | |
| CNBr | — | — | — | M | — | — |
| Enterokinase | D or E | D or E | D or E | K | — | — |
| Factor Xa | A, F, G, I, L, T, V or | D or E | G | R | — | — |
| | M | | | | | |
| Formic acid | — | — | — | D | — | — |
| Glutamyl | — | — | — | E | — | — |
| endopeptidase | | | | | | |
| GranzymeB | I | E | P | D | — | — |
| Hydroxylamine | — | — | — | N | G | — |
| Iodosobenzoic acid | — | — | — | W | — | — |
| LysC | — | — | — | K | — | — |
| Neutrophil elastase | — | — | — | A or V | — | — |
| NTCB (2-nitro-5- | — | — | — | — | C | — |
| thiocyanobenzoic | | | | | | |
| acid) | | | | | | |
| Pepsin (pH 1.3) | — | not H, K, or R | not P | not R | F or L | not P |
| | — | not H, K, or R | not P | F or L | — | not P |
| Pepsin (pH >2) | — | not H, K or R | not P | not R | F, L, W or Y | not P |
| | — | not H, K or R | not P | F, L, W or | — | not P |
| | | | | Y | | |
| Proline-endopeptidase | — | — | H, K or R | P | not P | — |
| Proteinase K | — | — | — | A, E, F, I, L, | — | — |
| | | | | T, V, W or | | |
| | | | | Y | | |
| Staphylococcal | — | — | not E | E | — | — |
| peptidase I | | | | | | |
| Thermolysin | — | — | — | not D or E | A, F, I, L, M or | — |
| | | | | | V | |
| Thrombin | — | — | G | R | G | — |
| | A, F, G, I, L, T, V or | A, F, G, I, L, T, | P | R | not D or E | not |
| | M | V, W or A | | | | DE |
| Trypsin | — | — | — | K or R | not P | — |
| | — | — | W | K | P | — |
| | — | — | M | R | P | — |

The ratio of modified ECM material to unmodified ECM material may be optimized based on the application. For instance, the ratio of modified ECM material to unmodified ECM material may be optimized based on such parameters as material properties and desired cell attachment. In some embodiments, the ratio of modified ECM material to unmodified ECM material may be about 5:1 to about 1:5 (e.g. about 5:1, about 4:1, about 3:1, about 2:1, about 1:1, about 1:2, about 1:3, about 1:4, or about 1:5). The modified ECM material may have a degree of modification of about 5 to about 95 percent (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%). In some cases, the modification may be (meth)acrylation of the ECM. In these embodiments, the ratio of (meth)acrylated ECM material to non-(meth)acrylated ECM material may be about 5:1 to about 1:5 (e.g. about 5:1, about 4:1, about 3:1, about 2:1, about 1:1, about 1:2, about 1:3, about 1:4, or about 1:5). The (meth)acrylated ECM material may have a degree of (meth)acrylation of about 5 to about 95 percent (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%).

The composition may include a photo initiator. The photo initiator is not particularly limited. The photo initiator may be a photoactive dye. The photoactive dye may be a UV dye with absorbance spectra between 100-420 nm. The UV dye may have an absorbance spectra between 300 nm to 420 nm. The photoactive dye may have a wavelength range of 300 nm to 400 nm. The photoactive dye may be non-cytotoxic. The photoactive dye may include a benzyne ring in the molecular structure. The photoactive dye may be quinolone yellow, a UV dye, or a dye with a molecular structure similar thereto. The photoactive dye may be UV 386A dye.

Photo initiators, may include, for example, benzophenone, phenyl bis (2,4,6-trimethylbenzoyl) phosphine oxide (BAPO), 2-hydroxy-2-methyl-1-phenyl-propan-1-one, 2-hydroxy-4′-(2-hydroxyethoxy)-2-methylpropiophenone, 2,2′-azobis[2-methyl-n-(2-hydroxyethyl)propionamide], 2,2-dimethoxy-2-phenylacetophenone, lithium phenyl(2,4,6-trimethylbenzoyl) phosphinate (LAP), and ethyl (2,4,6-trimethylbenzoyl) phenylphosphinate, Sodium phenyl-2,4,6-trimethylbenzoylphosphinate (NAP), Trimethylbenzoyl based photoinitiators, diphenyl(2,4,6-trimethylbenzoyl)phosphine oxide (TPO nanoparticle) Irgacure class of photoinitiators, ruthenium, and riboflavin, or mixtures thereof.

Methods

In certain embodiments, disclosed is a hydrogel scaffold, object, and/or method of manufacture. The object may be formed by 3D printing. The compositions and materials listed above may be used as ink in a 3D printer to form the 3D printed object.

The skilled artisan would appreciate the methods of printing known in the art, and non-limiting examples include selective laser sintering (SLS) method, a fused deposition modeling (FDM) method, a 3D inkjet printing method, a digital light processing (DLP) method, and a stereolithography method. In the fused deposition modeling (FDM) method, the inks are deposited by an extrusion head, which follows a tool-path defined by a CAD file. The materials are deposited in layers as fine as 25 μm thick, and the part is built from the bottom up, one layer at a time. Some 3D printers based on the fused deposition modeling method are equipped with dual print nozzle heads that can extrude two different materials, one being a building material and the other being a support, such as a pillar, material. The support material can be washed with water.

3D inkjet printing is effectively optimized for speed, low cost, high resolution, and ease-of-use, making it suitable for visualizing during the conceptual stages of engineering design through to early-stage functional testing. Complicated 3D articles in the ink-jet printing method are produced from ink compositions by jetting followed by UV/Vis light. The photo-curable ink in the ink-jet printing process may be jetted through several nozzles on the building platform with a pattern defined by a CAD file.

An efficient technology among 3D printing technologies is a digital light process (DLP) method or stereolithography (SLA). In a 3D printer using the DLP or SLA method, the ink material is layered on a vat or spread on a sheet, and a predetermined area or surface of the ink is exposed to ultraviolet-visible (UV/Vis) light that is controlled by a digital micro-mirror device or rotating mirror. In the DLP method, additional layers are repeatedly or continuously laid and each layer is cured until a desired 3D article is formed. The SLA method is different from the DLP method in that ink is solidified by a line of radiation beam. Other methods of 3D printing may be found in 3D Printing Techniques and Processes by Michael Degnan, December 2017, Cavendish Square Publishing, LLC, the disclosure of which is hereby incorporated by reference.

In some embodiments, once the 3D printed object is formed, cells are deposited on it.

### EXAMPLES

The following example describes specific aspects of some embodiments of this disclosure to illustrate and provide a description for those of ordinary skill in the art. The example should not be construed as limiting this disclosure, as the example merely provides specific methodology useful in understanding and practicing some embodiments of this disclosure.

### Printing

All samples used in the examples were prepared using a 3D inkjet printing method or a digital light processing (DLP) method, wherein the components of the bioink were mixed, and the 3D object was then printed using, e.g., a Labfab inverted digital light projection (DLP) 3D printing system.

Components of the bioink were procured from commercial sources when available. Biologically active peptides were synthesized by performing Michael-type reactions to link the peptide to one or more (meth)acrylate monomer or polymer.

### Cell Adhesion Investigations

The following cell adhesion tests were performed by following at least a portion of the following protocol. First, 3D-printed disks were obtained, and if made with collagen that is not fully methacrylated, then the disk was subjected to crosslinking treatment with sterile 1M NaHCO₃. The disks were washed twice for at least 30 minutes with DPBS++. The disks were then placed in 5× Anti-Anti solution (100× Antibiotic Antimycotic diluted in DPBS−−) overnight. The 5× Anti-anti was then swapped out for 2 PBS washes for at least 30 minutes each. Using an optical 96 well plate, 3 wells were filled with 200 uL of wash each. The average 384 nm absorbance of the washes from those 3 wells were taken using the SpectraMax i3x (or equivalent). The 384 nm absorbance was less than 0.1 indicating that trace dye/PI is at an acceptable level.

The disk was then transferred to a well with 500 uL of LFN GM (Lung Fibroblasts growth medium) and a predetermined number of cells are added to the solution. After the predetermined amount of time, 300 uL/well of 10% Formalin+0.1% TritonX100 to controls and 500 uL/well was added to the wells containing 3D disks and incubated for 15 minutes at room temperature. The fixative was then aspirated into a waste container, and the samples were washed with DPBS−− (3×5 minute washes). The disks were then stained with a solution containing 1:20,000 SytoxOrange and 3:400 Phalloidin 488. After a final DPBS wash, the disks were assessed for cell adhesion.

### Example 1

Lung fibroblasts were cultured for 7 days on glass (control), bioink containing 50% DOF collagen, bioink containing 90% DOF collagen, and bioink containing 0% DOF collagen and 90% DOF collagen (1:2 ratio).

Bioinks were formulated by the above procedures. Bioinks comprising collagen with varying degrees of functionalization were then 3D printed into disks. Lung fibroblasts were deposited on each sample according to the above procedures. The fibroblasts were cultured for 7 days on glass (control), a printed disk from a bioink containing 50% DOF collagen, a printed disk from a bioink containing 90% DOF, and a printed disk from a hybrid bioink formula containing a hybrid of 0% DOF collagen and 90% DOF collagen (1:2 ratio). Images were taken as shown in FIG. 2(A). A graph of cell spread, density, and % cell coverage was plotted for each sample as shown in FIG. 2(B) graph of cell spread, density, and % cell coverage.

### Example 2

Pulmonary artery endothelial cells were cultured for 1 day on glass, a printed disk from a 9% PEGDA Hybrid, and a printed disk from a 9% PEGDA.

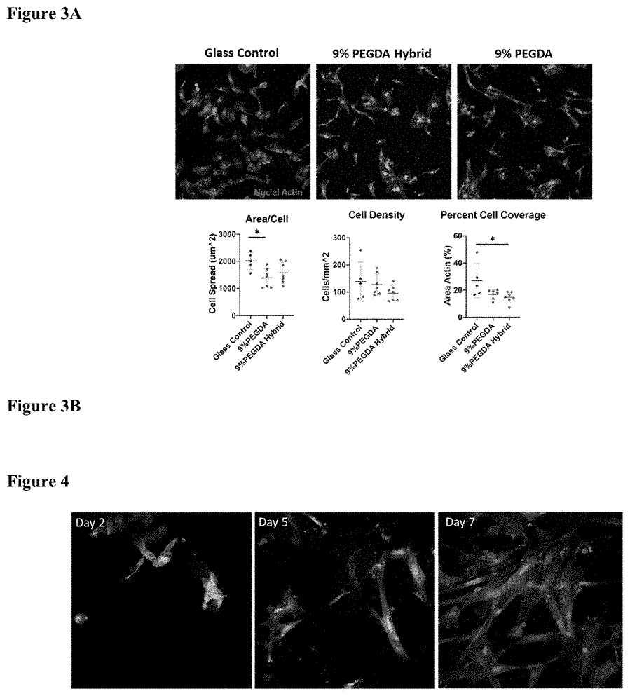

Bioinks were formulated by the above procedures. Bioinks comprising PEGDA and PEGDA hybrid were 3D printed into disks. Pulmonary artery endothelial cells were deposited on each sample according to the above procedures. The pulmonary artery endothelial cells were then cultured for 1 day on glass (control), PEGDA, and PEGDA hybrid disks. Images were taken as shown in FIG. 3(A). A graph of cell spread, density, and % cell coverage was plotted for each sample as shown in FIG. 3(B) graph of cell spread, density, and % cell coverage.

### Example 3

Lung smooth muscle cells ability to attach and proliferate on a 3D printed disk comprising PEGDA MW 3400, a 3D printed disk comprising 95% DOF collagen and 0% DOF collagen (2:1 ratio) was investigated.

Bioinks were formulated by the above methods. The bioink was 3D printed to form a disk comprising PEGDA MW 3400, a 3D printed disk comprising 95% DOF collagen and 0% DOF collagen (2:1 ratio) Lung smooth cells were deposited on the disk according to the above methods. The cells were allowed to attach, proliferate and spread across the disk over the course of 7 days. Images of the disk as days 2, 5, and 7 were taken as shown in FIG. 4.

### Example 4

The number of cell adhesion properties to a 3D printed object made from bioinks with different ratios of functionalized and non-functionalized collagen. Bioink C201, C202, and C203 were formed comprising the components in Table 2 below. These bioinks were 3D printed according to the above methods.

TABLE 2

| Category | Component | 201 | 202 | 203 |
|---|---|---|---|---|
| Collagen | ColMA | 20-30 | 20-30 | — |
| | NM Col | 50-60 | 60-65 | 80-90 |
| Monomer | SR9035 | 1-4 | 2-5 | 8-11 |
| | PEG600DM | 1-2 | 0.3-1 | — |
| | HEAA | 12-16 | 11-15 | 3-6 |

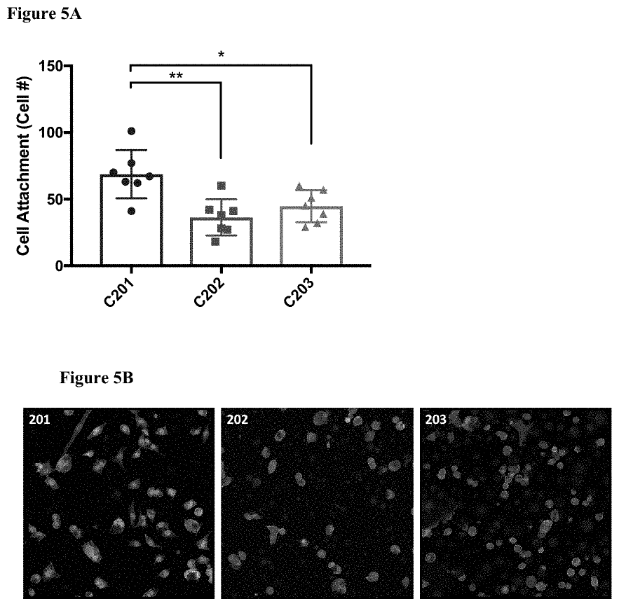

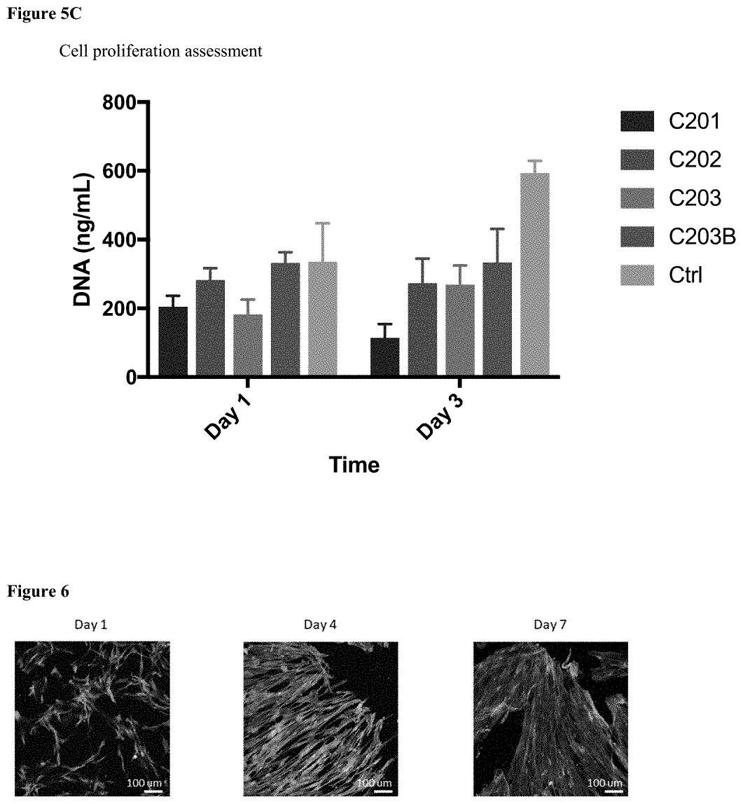

After printing, cells were deposited on each object according to the above methods. The cell adhesion was measured of each sample C201, C202, C203 and plotted on FIG. 5A. FIG. 5B shows images of the samples.

As shown in the graph 5C, sample C201 showed significantly greater cell adhesion over C202 and C203.

### Example 5

A 3D printed bioink containing functionalized and non-functionalized collagen was examined to evaluate support for lung fibroblast cell attachment and proliferation by day.

A hybrid bioink comprising 30 DOF Hybrid Collagen (mixture of 0 DOF (Non Methacrylated Collagen) and 90 DOF (Methacrylated Collagen)) was formed by adding LAP as solid (0.5-2 wt. %) to an aqueous solution comprising PEGDA 3400 (5-14 wt. %) and HPA (8-17 wt. %), and the resulting solution was speed mix until clear. Dye (0.12%) was added, and the solution was confirmed to be between a pH of 2-3. Next, the 30 DOF Hybrid Collagen (40-55 wt. %) was added, and the solution was stirred until homogenous. The degree of functionalization was 30%. The bioink was 3D printed. Lung cells were deposited on the sample according to the above methods. The amount of cell attachment and proliferation was imaged on days 1, 4, and 7 and shown in FIG. 6.

### Example 6

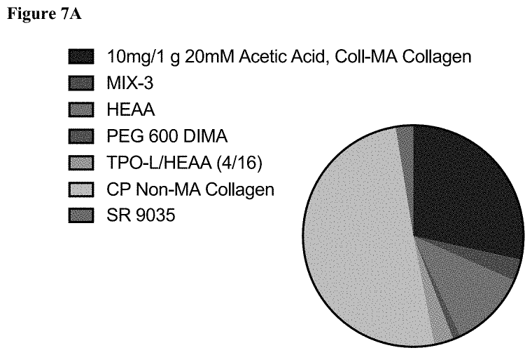

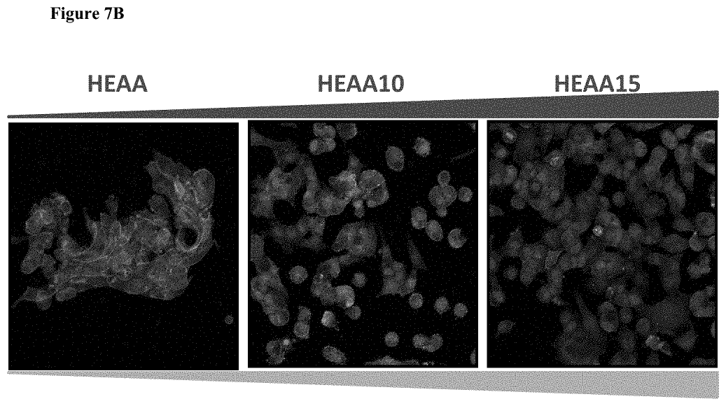

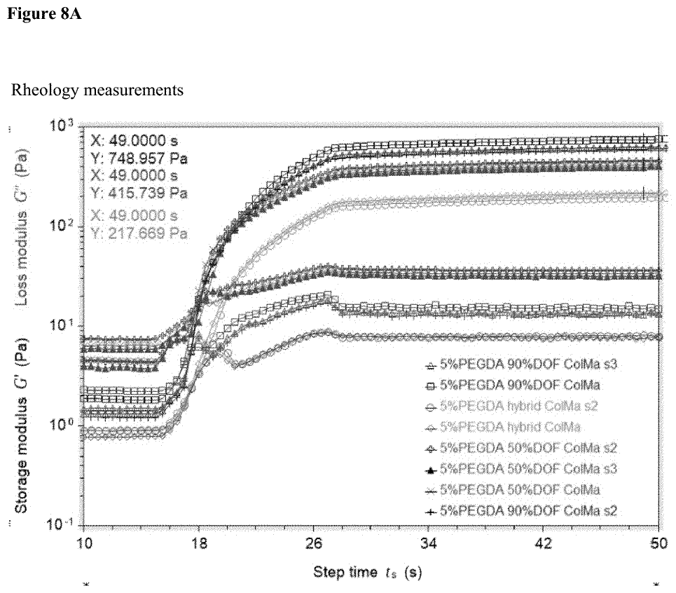

Effect of HEAA (%) component on cell adhesion properties were examined. 3D-printed disks described in FIG. 7 were obtained according to the above methods, and the cells were deposited on the disks according to the above methods.

The effect of the HEAA (%) component on cell adhesion can be seen in FIG. 7. FIG. 7A shows the ratio of components in the bioink. Samples including 5%, 10%, and 20% HEAA were tested, and the cell adhesion is shown in FIG. 7B.

### Example 7

Effects of CollMA DOF on cell adhesion and proliferation (>90%, ˜50%, hybrid (50% Non-MA, 50% HM). Samples were formed by 3-D printing bioinks comprising compositions of Samples below.

### Sample 1 Composition

### PEGDA3.4k (3-10 wt. %); LAP (0.3-1.0 wt. %); UV386A; ColMA (90 DOF)

### Sample 2 Composition

### PEGDA3.4k (3-10 wt. %); LAP (0.3-1.0 wt. %); UV386A; ColMA (50 DOF)

### Sample 3 Composition

PEGDA3.4k (3-10 wt. %); LAP (0.3-1.0 wt. %); UV386A; ColMA hybrid (0 DOF+90 DOF)

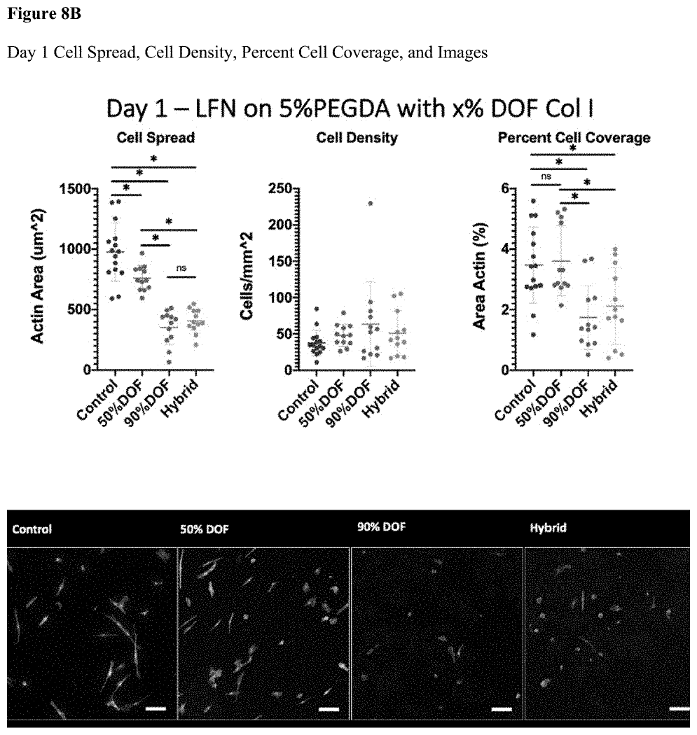

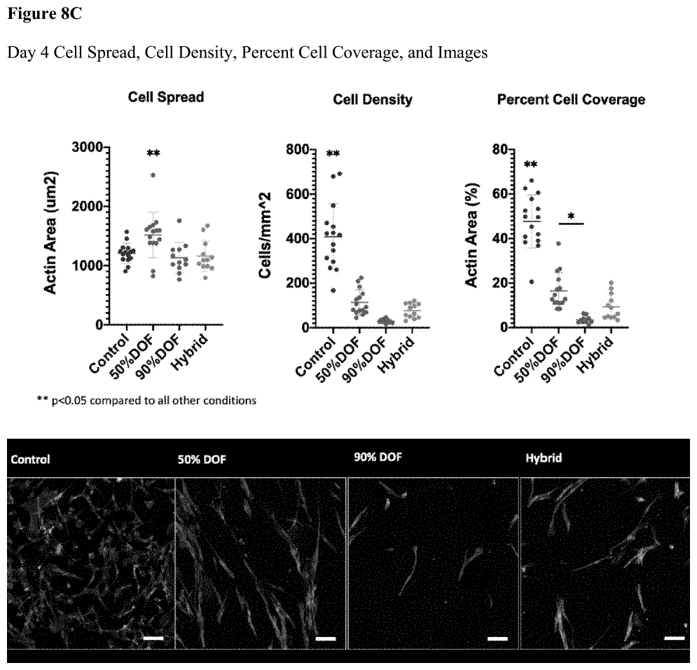

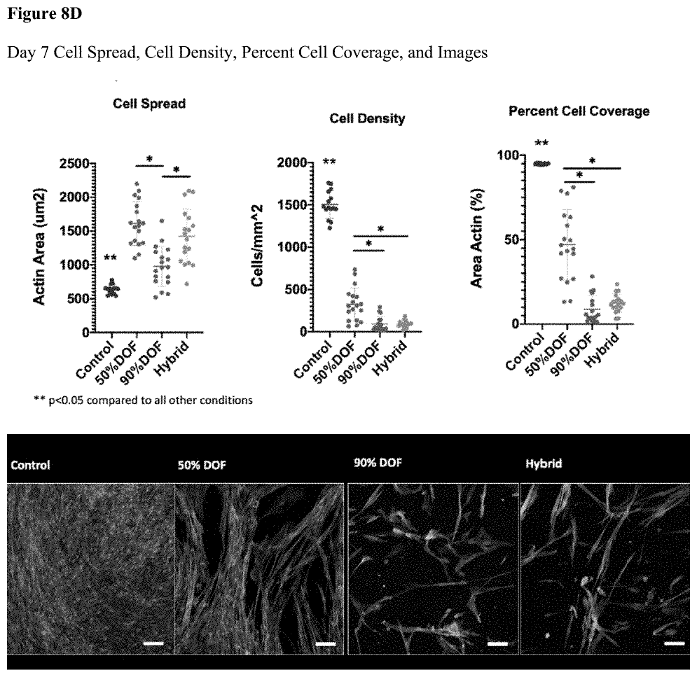

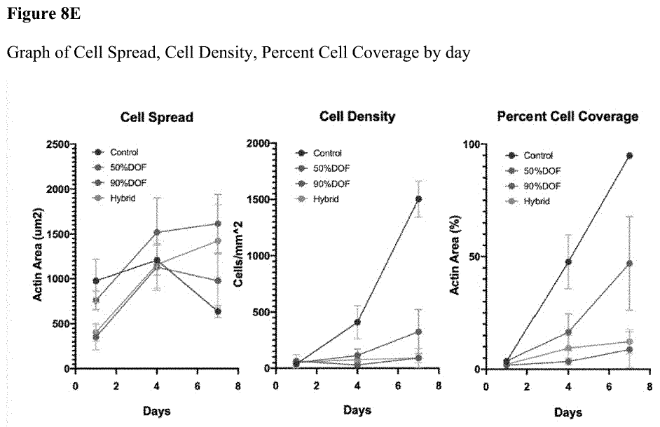

The Rheology was measured. Results are shown in the graph of FIG. 8A.

The bioinks were 3D printed using a Labfab inverted digital light projection (DLP) 3D printing system. Fibroblasts were deposited and incubated by procedures described above. FIG. 8E shows a graph of cell spread, cell density, and percent cell coverage by day. The cell spread, cell density, and percent coverage were calculated by procedures described above on days 1, 4, and 7 as shown in FIG. 8B-D. A graph of cell spread, cell density, and percent cell coverage between the samples were graphed in FIG. 8E. As shown, cell spread was improved on hybrid and 50% DOF inks by day 7. Cells were actively proliferating on 50% DOF inks over the course of 7 days compared to 90% DOF and Hybrid inks. One model for this is that unmethacrylated collagen may leach out of the hybrid gels.

### Example 8

Comparison of Cell Density, Cell Spread, and Cell Coverage of PAEC cells.

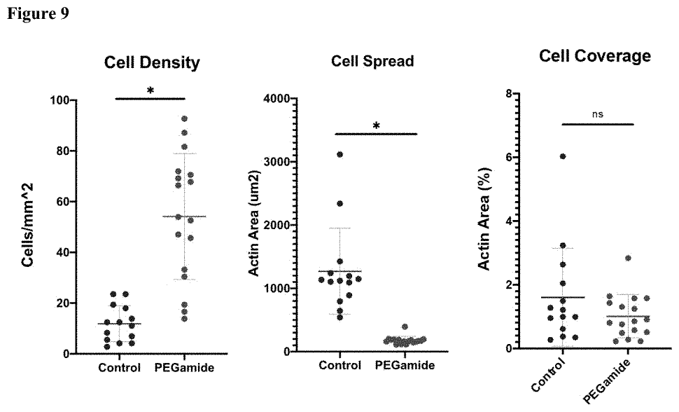

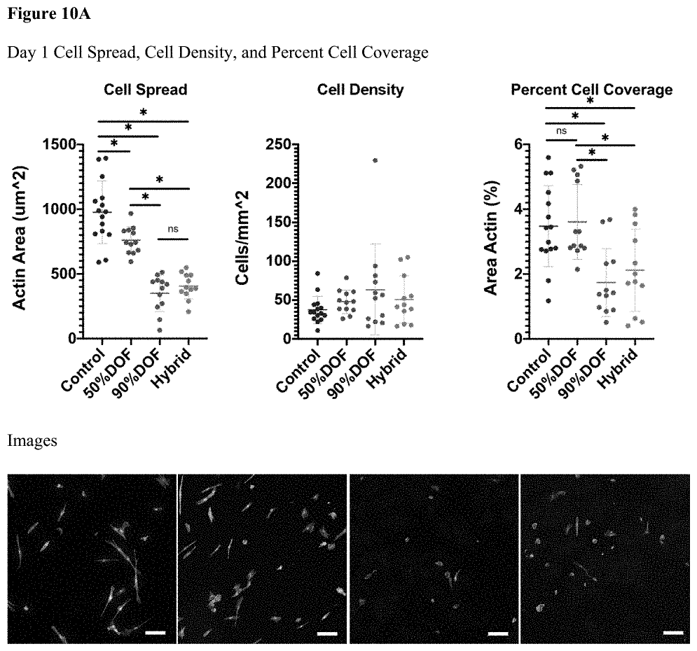

PAEC cells were seeded at an amount of 10,500/cm² and cultured for 1 day. The sample was compared to a glass slide. As shown in FIG. 9, the cell density, cell spread, and cell coverage was compared between sample and control.

### Example 9

Samples with components like Samples 1-3, above, were produced using 5 different batches of Collagen. For each of these batches samples were formed by mixing the ink components in water and then 3D printing the ink into disks. Each sample was seeded with 5000 cells/cm² (PAEC). Additionally, a glass control was seeded with PARC. Cell spread, cell density, percent cell coverage were assessed at days 1, 4, and 7.

### Example 10

LFN on 5% PEGDA with x % DOF Col I

Samples of 5% PEGDA and Collagen I with varying degrees of functionalization were produced. The samples included a glass control, 50% degree of Collagen I functionalization, 90% of Collagen I functionalization, and a hybrid sample. The samples were formed by mixing the ink components in water and then 3D printing the ink into disks. Each sample was seeded with cells in a similar fashion to what is described above.

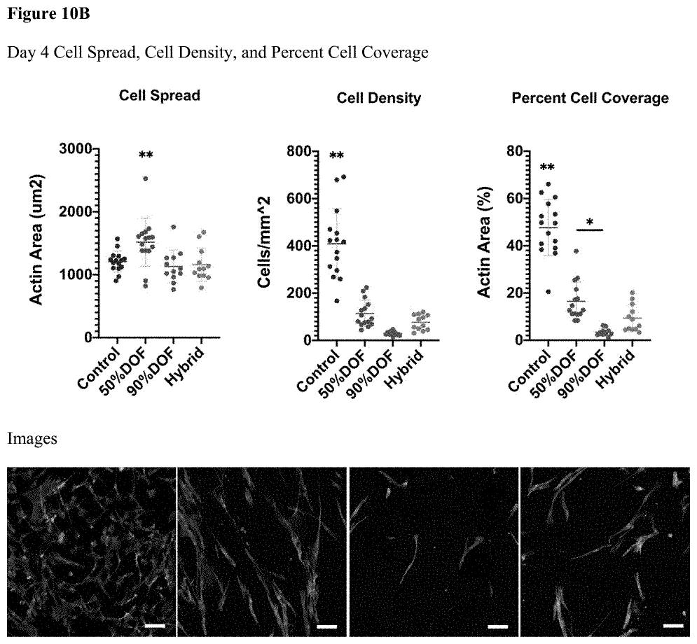

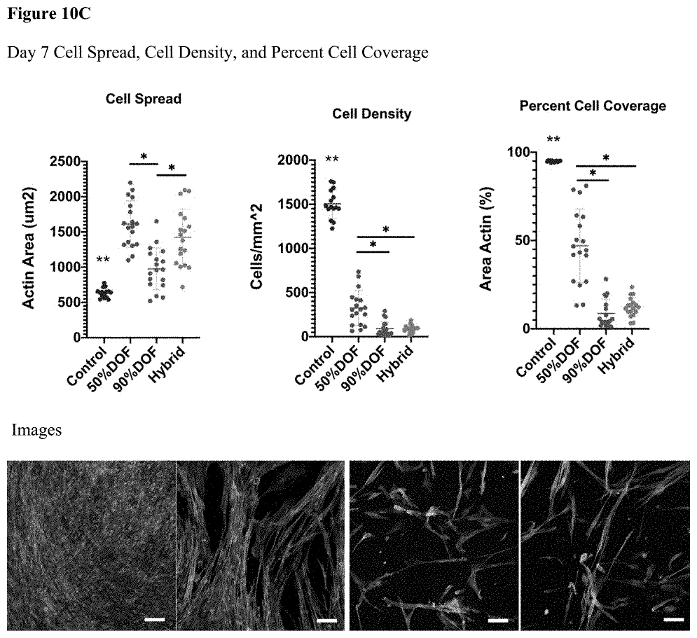

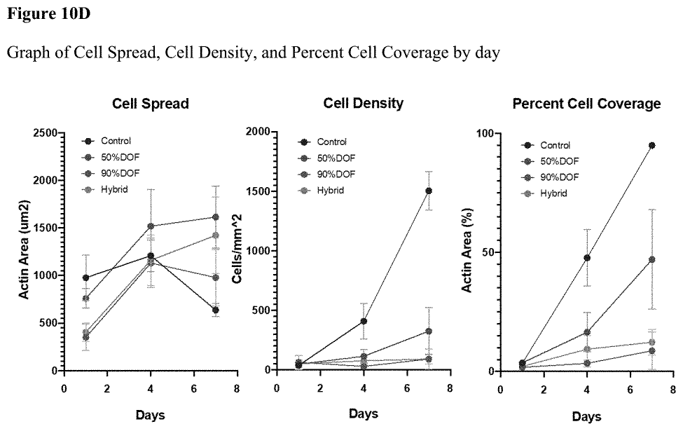

As shown in FIG. 10A, cell spread, cell density, percent cell coverage and images were compared between each sample on day 1. As shown in FIG. 10B, cell spread, cell density, percent cell coverage and images were compared between each sample on day 4. As shown in FIG. 10C, cell spread, cell density, percent cell coverage and images were compared between each sample on day 7. As shown in FIG. 10D, the cell spread, cell density, and percent cell coverage were graphed by day for each sample.

### Example 11

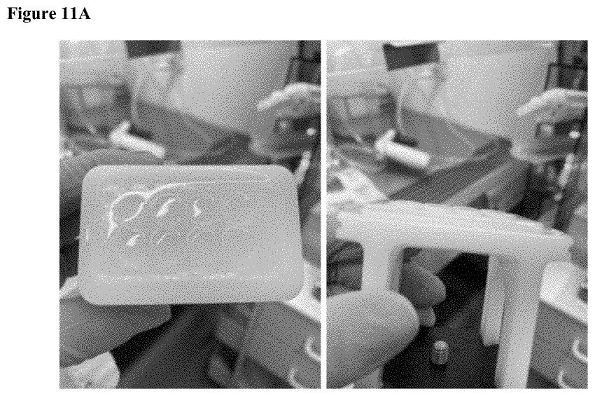

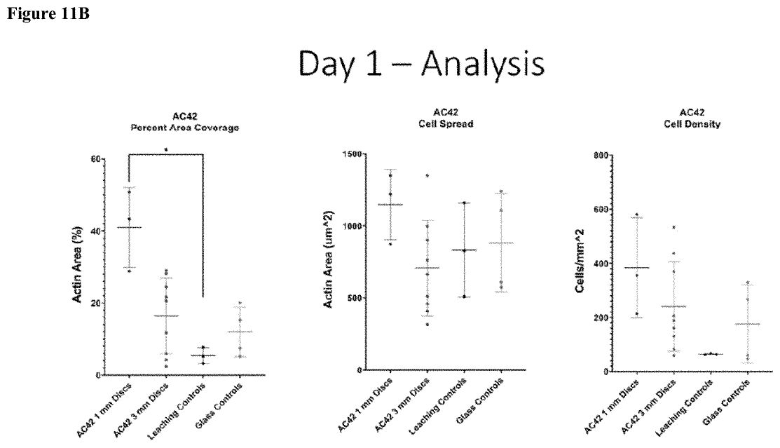

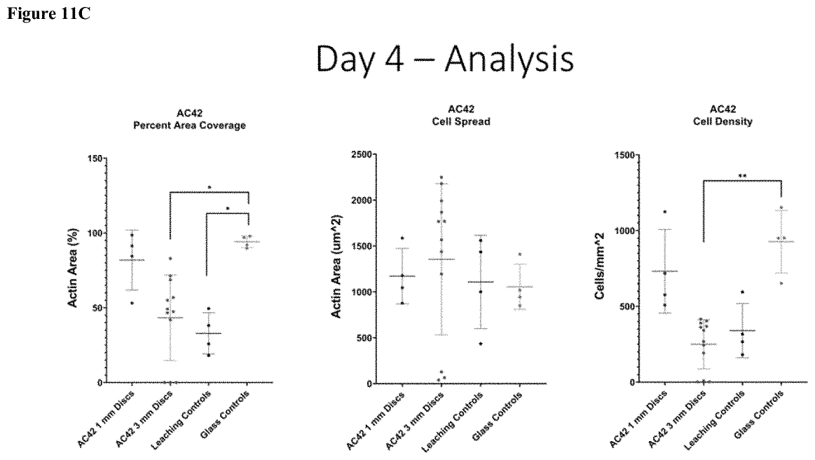

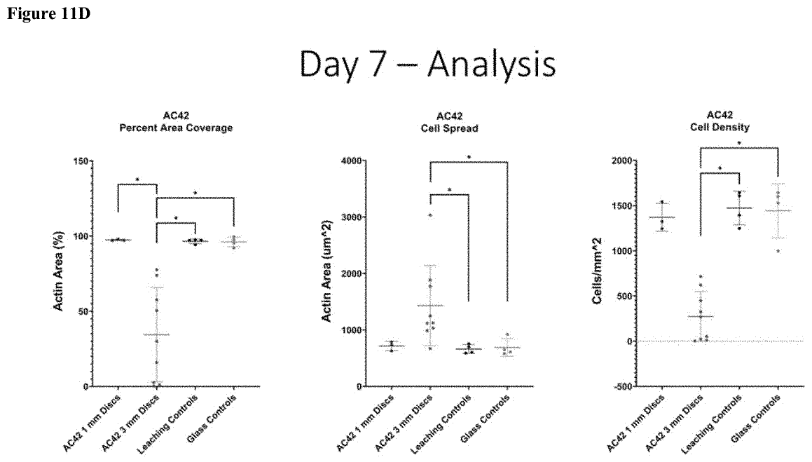

This study assessed the biocompatibility of AC42 (bioink with 6-12 wt. % HPA; 6-12 wt. % PEGDA 3.4k; various Methacrylated Collagen; LAP, UV dye). The bioink was printed on a LabFab printer to form 3 mm disks and 1 mm disks for a three time point study (Day 1, 4, 7). These disks were adhered to the platform. Images of the disks prior to seeding cells are shown in FIG. 11A.

The disks were crosslinked in 1M NaHCO₃ for 10 min in 24 well plates. Following crosslinking, the disks were transferred to a petri dish for PBS++ washes (2 times, at least 10 min each) on the shaker plate at speed 60. Disks were transferred to well plates for a 5× wash overnight.

LFN cells were seeded on each disk. 10K cells were added to each well to seed each sample disk. 20K cells per well were added to seed the controls. As shown in FIG. 11B, cell spread, cell density, percent cell coverage and images were compared between each sample on day 1. Cells were spreading well by day 1 (getting very confluent). As shown in FIG. 11C, cell spread, cell density, percent cell coverage and images were compared between each sample on day 4. Cells were completely confluent by day 4. As shown in FIG. 11D, cell spread, cell density, percent cell coverage and images were compared between each sample on day 7. Cells were overlapping each other by day 7. The disks had great cell attachment, but some peeling off of confluent cell sheets by day 4 and day 7 so the cell density for LFN studies may be decreased.

### Example 12

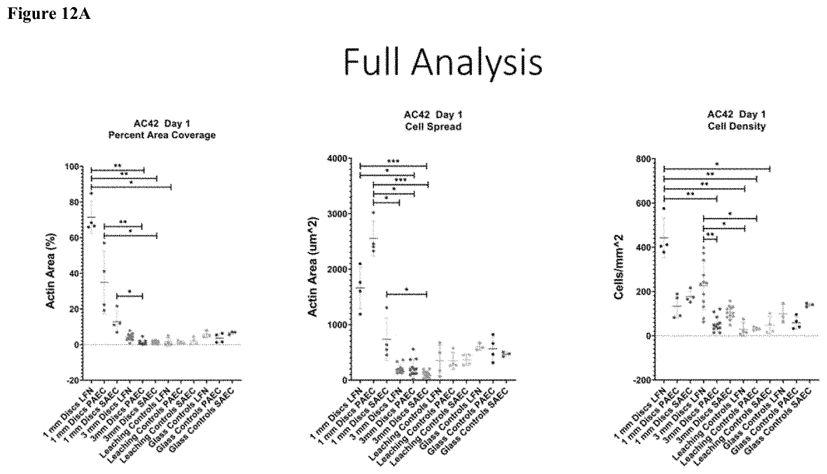

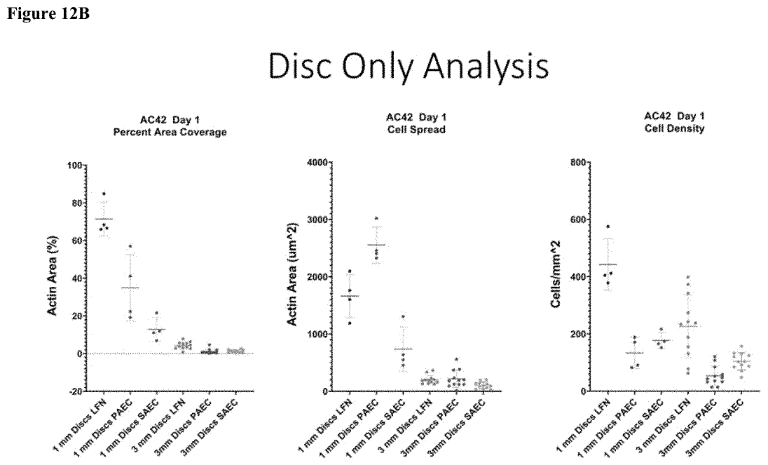

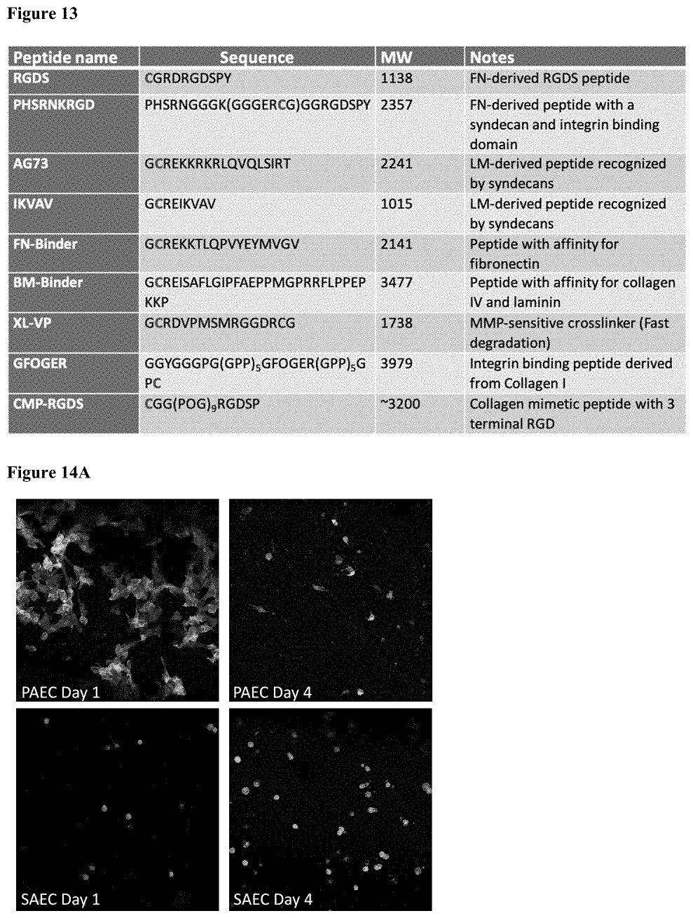

FIGS. 12A-12B show a comparison of Percent Area Coverage, Cell Spread, and Cell Density Comparison of AC42 1 mm disks, 3 mm disks, Leaching Controls, and Glass Controls seeded with LFN, PAEC or SAEC seeds. The AC42 bioink was printed on a LabFab printer to form 3 mm disks and 1 mm disks for a three time point study (Day 1, 4, 7). These disks were adhered to the platform. Images of the disks are shown in FIG. 12A.

The disks were crosslinked in 1M NaHCO₃ for 10 min in 24 well plates. Following crosslinking, the disks were transferred to a petri dish for PBS++ washes (2 times, at least 10 min each) on the shaker plate at speed 60. Disks were transferred to well plates for a 5× wash overnight. LFN, PAEC or SAEC cells were seeded on each disk.

FIG. 12A shows a comparison on day 1 of Percent Area Coverage, Cell Spread, and Cell Density Comparison of AC42 1 mm disks, 3 mm disks, Leaching Controls, and Glass Controls seeded with LFN, PAEC or SAEC seeds. FIG. 12B shows a comparison on day 1 of Percent Area Coverage, Cell Spread, and Cell Density Comparison of AC42 1 mm disks and 3 mm disks seeded with LFN, PAEC or SAEC seeds.

### Example 13

Disks were printed from bioinks comprising 2-5 wt. % HPA or HBA, 1-5 wt. % PEGDA575, 3-8 wt. % PEGDA6000, 1-6 mM PEG₃₄₀₀AcrylateCGRGDS, LAP, UV386A and water.

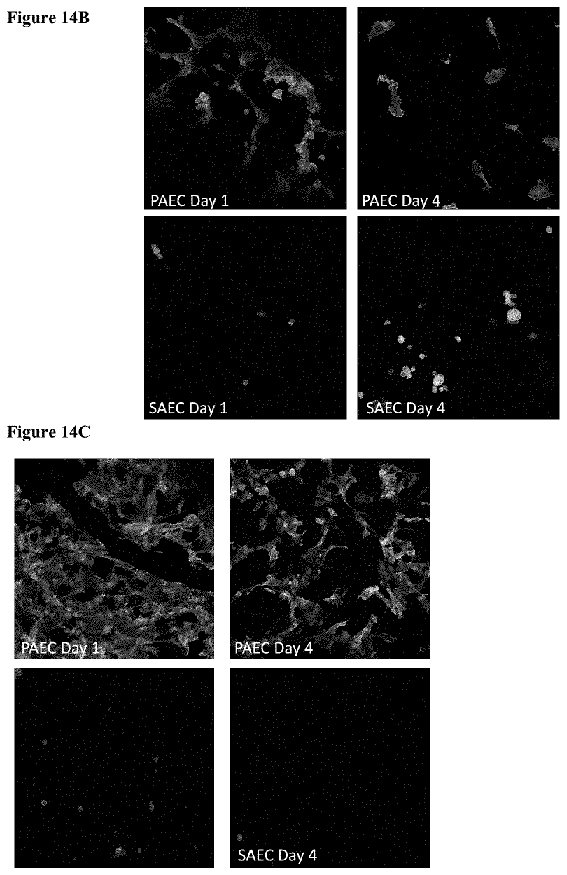

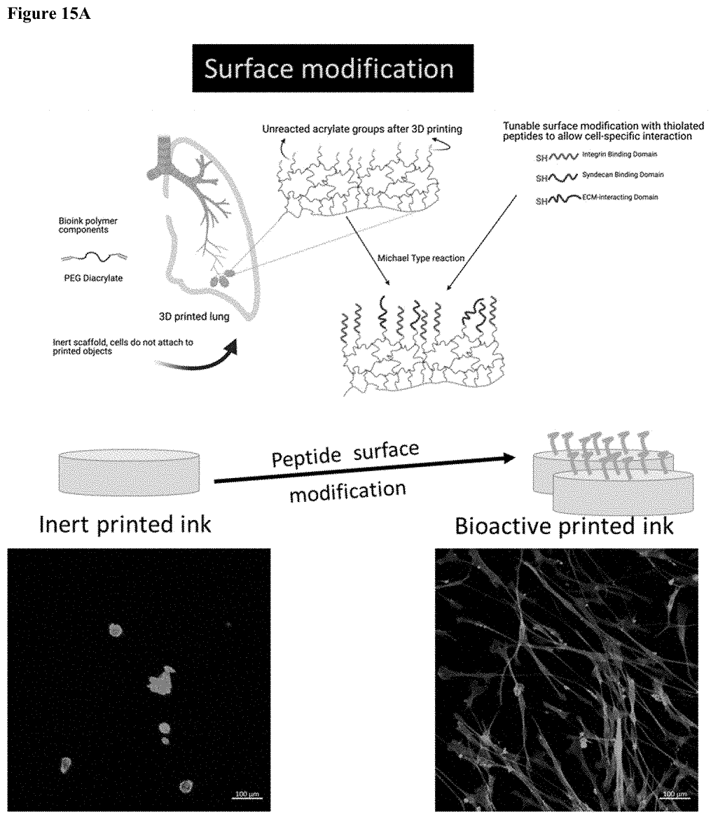

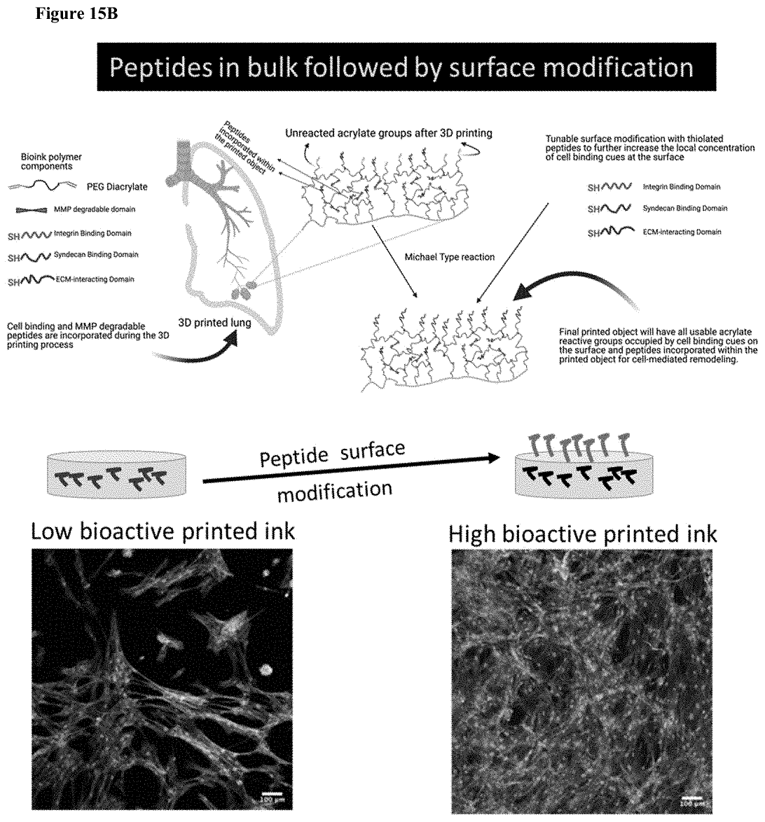

PAEC and SAEC coverage was examined on days 1 and 4. FIG. 14A shows cell coverage on a disk comprising 2-5 wt. % HPA, 1-2 wt. % PEGDA575, 3-8 wt. % PEGDA6000, 1-6 mM PEG₃₄₀₀AcrylateCGRGDS, LAP, UV386A. FIG. 14B shows cell coverage on a disk comprising 2-5 wt. % HPA, 3-4 wt. % PEGDA575, 3-8 wt. % PEGDA6000, 1-6 mM PEG₃₄₀₀AcrylateCGRGDS, LAP, UV386A. FIG. 14C shows cell coverage on a disk comprising 2-5 wt. % HBA, 3-4 wt. % PEGDA575, 3-8 wt. % PEGDA6000, 1-6 mM PEG₃₄₀₀AcrylateCGRGDS, LAP, UV386A.

### Example 14

Disks printed from inks having the following components were surface modified with PHSRNKRGDS (0.5-1 mM) and AG73 (0.3-0.7 mM) by reacting unreacted acrylate groups after printing with the peptides in solution.

| Reagent | AA42 | AC51 |
|---|---|---|
| PEGDA 3400 | 5-10% | 0.5-2% |
| PEGDA 6000 | | 3-6% |
| HPA | 8-15% | 8-15% |
| LAP | 1-3% | 1-3% |
| UV386 | 0.1-0.2% | 0.1-0.2% |

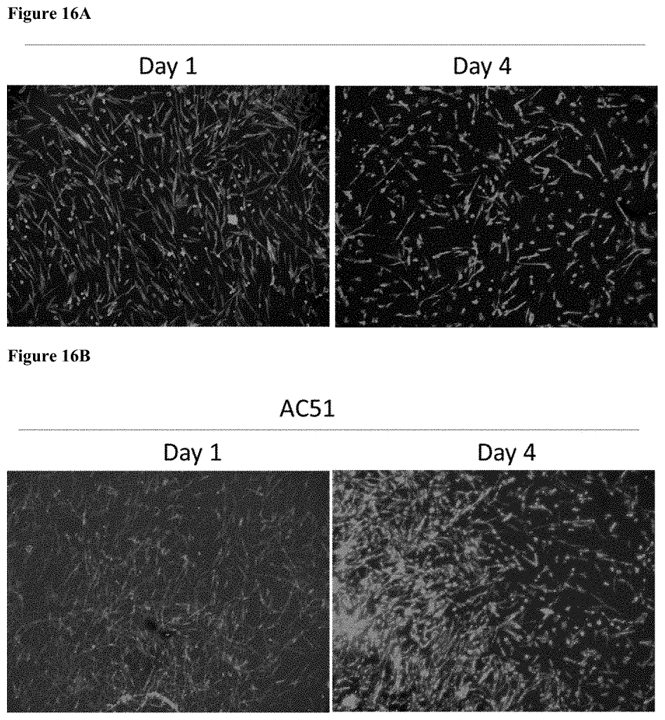

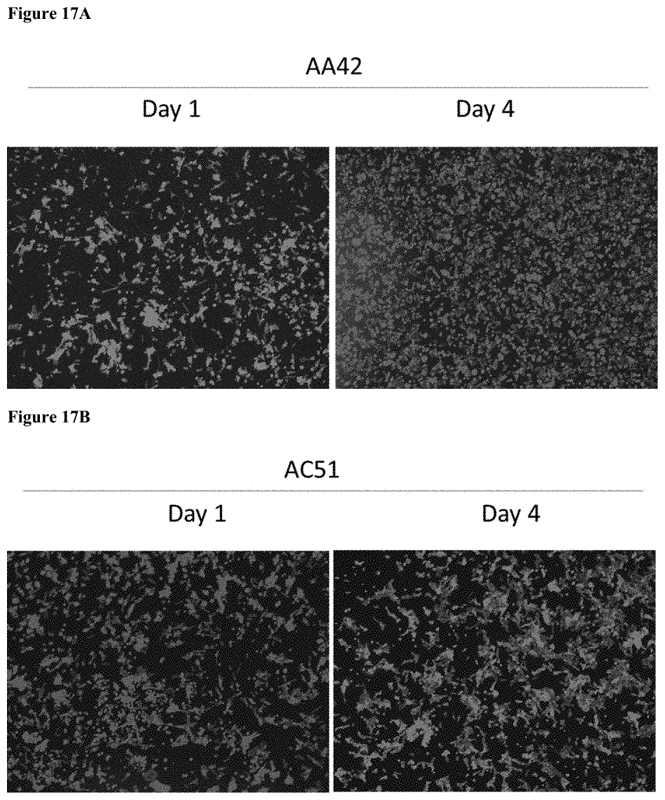

Growth/attachments of Fibroblasts were examined on day 1 and 4, as shown in FIGS. 16A-16B. Growth/attachments of SAEC were examined on day 1 and 4, as shown in FIGS. 17A-17B.

As used herein, the singular terms “a,” “an,” and “the” may include plural referents unless the context clearly dictates otherwise. Thus, for example, reference to an object may include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claim(s). In addition, many modifications may be made to adapt a particular situation, material, composition of matter, method, operation or operations, to the objective, spirit and scope of the disclosure. All such modifications are intended to be within the scope of the claim(s) appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

1. A composition comprising a cross-linked (meth)acrylated extracellular matrices (ECM) material and a non-(meth)acrylated ECM material.

2. The composition of claim 1, wherein a ratio of (meth)acrylated ECM material to non-(meth)acrylated ECM material is about 5:1 to about 1:5.

3. The composition of claim 1, wherein the (meth)acrylated ECM material has a degree of (meth)acrylation of about 5 to about 95 percent.

4. The composition of claim 1, wherein the ECM material is selected from collagen, gelatin, elastin, and fibronectin.

5. The composition of claim 4, wherein the ECM material is collagen I.

6. The composition of claim 1, wherein the (meth)acrylated ECM material includes a mono- or di-(meth)acrylated ECM or ECM-like material.

7. The composition of claim 6, wherein the ECM or ECM-like material is selected from RGD, KQAGDV (SEQ ID NO: 1), YIGSR (SEQ ID NO: 2), REDV (SEQ ID NO: 3), IKVAV (SEQ ID NO: 4), RNIAEIIKDI (SEQ ID NO: 5), KHIFSDDSSE (SEQ ID NO: 6), VPGIG (SEQ ID NO: 7), FHRRIKA (SEQ ID NO: 8), KRSR (SEQ ID NO: 9), APGL (SEQ ID NO: 10), VRN, AAAAAAAAA (SEQ ID NO: 11), GGLGPAGGK (SEQ ID NO: 12), GVPGI (SEQ ID NO: 13), LPETG(G)n (SEQ ID NO: 14), and IEGR (SEQ ID NO: 15).

8. The composition of claim 6, wherein the ECM or ECM-like material is a sequence sensitive to a protease.

9. The composition of claim 8, wherein the protease is selected from Arg-C proteinase, Asp-N endopeptidase, BNPS-Skatole, Caspase 1-10, Chymotrypsin-high specificity (C-term to [FYW], not before P), Chymotrypsin-low specificity (C-term to [FYWML (SEQ ID NO: 16)], not before P), Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin, Proline-endopeptidase, Proteinase K, Staphylococcal peptidase I, Thermolysin, Thrombin and Trypsin.

10. The composition of claim 1, wherein the composition further comprises a polymerized poly(ethylene glycol) di-(meth)acrylate, poly(hydroxy ethyl) (methacrylate), Poly N-hydroxyacrylamide, 3-hydroxypropyl acrylate, Hydroxy butyl acrylate.

11. The composition of claim 10, wherein the poly(ethylene glycol) di-(meth)acrylate monomer has a weight average molecular weight (M_{w}) of about 400 to about 20,000.

12. The composition of claim 10, wherein the poly(ethylene glycol) di-(meth)acrylate monomer has a M_{w} of about 2000 to about 4000.

13. The composition of claim 10, wherein the polymerized poly(ethylene glycol) di-(meth)acrylate monomer is present in an amount of about 5 to about 50 wt. % of the composition.

14. The composition of claim 1, wherein the composition supports primary cell and/or induced pluripotent stem cell attachment, proliferation, and spreading.

15. The composition of claim 1, wherein the composition is a molded or 3D printed hydrogel article.

16. The composition of claim 15, wherein the composition is a molded or 3D hydrogel printed article that has been photo-crosslinked.

17. The molded or 3D printed hydrogel article of claim 15, wherein the article is a three-dimensional article of an organ, wherein the organ is a mammalian organ.

18. A method of manufacturing a three-dimensional article comprising:
depositing a layer of a printable composition to a surface to obtain a deposited layer;
irradiating the deposited layer; and
repeating the depositing and irradiating steps until the deposited layers form the three-dimensional article;
wherein the printable composition comprises a (meth)acrylated extracellular matrices (ECM) material, a non-(meth)acrylated ECM material, and a photo initiator.

19. The method of claim 18, wherein the ECM material is selected from collagen, gelatin, elastin, and fibronectin.

20. The method of claim 1, wherein the printable composition further comprises a poly(ethylene glycol) di-(meth)acrylate monomer.

21-33. (canceled)


SEQUENCE LISTING (30 sequences)

SEQ ID NO: 1 - protein, 6 aa, Artificial Sequence
  Description of Artificial Sequence Synthetic peptide
KQAGDV

SEQ ID NO: 2 - protein, 5 aa, Artificial Sequence
  Description of Artificial Sequence Synthetic peptide
YIGSR

SEQ ID NO: 3 - protein, 4 aa, Artificial Sequence
  Description of Artificial Sequence Synthetic peptide
REDV

SEQ ID NO: 4 - protein, 5 aa, Artificial Sequence
  Description of Artificial Sequence Synthetic peptide
IKVAV

SEQ ID NO: 5 - protein, 10 aa, Artificial Sequence
  Description of Artificial Sequence Synthetic peptide
RNIAEIIKDI

SEQ ID NO: 6 - protein, 10 aa, Artificial Sequence
  Description of Artificial Sequence Synthetic peptide
KHIFSDDSSE

SEQ ID NO: 7 - protein, 5 aa, Artificial Sequence
  Description of Artificial Sequence Synthetic peptide
VPGIG

SEQ ID NO: 8 - protein, 7 aa, Artificial Sequence
  Description of Artificial Sequence Synthetic peptide
FHRRIKA

SEQ ID NO: 9 - protein, 4 aa, Artificial Sequence
  Description of Artificial Sequence Synthetic peptide
KRSR

SEQ ID NO: 10 - protein, 4 aa, Artificial Sequence
  Description of Artificial Sequence Synthetic peptide
APGL

SEQ ID NO: 11 - protein, 9 aa, Artificial Sequence
  Description of Artificial Sequence Synthetic peptide
AAAAAAAAA

SEQ ID NO: 12 - protein, 9 aa, Artificial Sequence
  Description of Artificial Sequence Synthetic peptide
GGLGPAGGK

SEQ ID NO: 13 - protein, 5 aa, Artificial Sequence
  Description of Artificial Sequence Synthetic peptide
GVPGI

SEQ ID NO: 14 - protein, 6 aa, Artificial Sequence
  Description of Artificial Sequence Synthetic peptide
LPETGG

SEQ ID NO: 15 - protein, 4 aa, Artificial Sequence
  Description of Artificial Sequence Synthetic peptide
IEGR

SEQ ID NO: 16 - protein, 5 aa, Artificial Sequence
  Description of Artificial Sequence Synthetic peptide
FYWML

SEQ ID NO: 17 - protein, 13 aa, Artificial Sequence
  Description of Artificial Sequence Synthetic peptide
PHSRNGGRGDSPY

SEQ ID NO: 18 - protein, 18 aa, Artificial Sequence
  Description of Artificial Sequence Synthetic peptide
GCREKKRKRLQVQLSIRT

SEQ ID NO: 19 - protein, 18 aa, Artificial Sequence
  Description of Artificial Sequence Synthetic peptide
GCREKKTLQPVYEYMVGV

SEQ ID NO: 20 - protein, 31 aa, Artificial Sequence
  Description of Artificial Sequence Synthetic polypeptide
GCREISAFLGIPFAEPPMGPRRFLPPEPKKP

SEQ ID NO: 21 - protein, 15 aa, Artificial Sequence
  Description of Artificial Sequence Synthetic peptide
GCRDGPQGWGQDRCG

SEQ ID NO: 22 - protein, 16 aa, Artificial Sequence
  Description of Artificial Sequence Synthetic peptide
GCRDVPMSMRGGDRCG

SEQ ID NO: 23 - protein, 6 aa, Artificial Sequence
  Description of Artificial Sequence Synthetic peptide
  MOD_RES (3)..(3): 4-hydroxyproline
GFXGER

SEQ ID NO: 24 - protein, 18 aa, Artificial Sequence
  Description of Artificial Sequence Synthetic peptide
LMNGCRDGPQGWGQDRCG

SEQ ID NO: 25 - protein, 7 aa, Artificial Sequence
  Description of Artificial Sequence Synthetic peptide
GGGERCG

SEQ ID NO: 26 - protein, 10 aa, Artificial Sequence
  Description of Artificial Sequence Synthetic peptide
CGRDRGDSPY

SEQ ID NO: 27 - protein, 17 aa, Artificial Sequence
  Description of Artificial Sequence Synthetic peptide
PHSRNGGGKGGRGDSPY

SEQ ID NO: 28 - protein, 9 aa, Artificial Sequence
  Description of Artificial Sequence Synthetic peptide
GCREIKVAV

SEQ ID NO: 29 - protein, 47 aa, Artificial Sequence
  Description of Artificial Sequence Synthetic polypeptide
  MOD_RES (26)..(26): 4-hydroxyproline
GGYGGGPGGPPGPPGPPGPPGPPGFXGERGPPGPPGPPGPPGPPGPC

SEQ ID NO: 30 - protein, 35 aa, Artificial Sequence
  Description of Artificial Sequence Synthetic polypeptide
  MOD_RES (5)..(5): 4-hydroxyproline
  MOD_RES (8)..(8): 4-hydroxyproline
  MOD_RES (11)..(11): 4-hydroxyproline
  MOD_RES (14)..(14): 4-hydroxyproline
  MOD_RES (17)..(17): 4-hydroxyproline
  MOD_RES (20)..(20): 4-hydroxyproline
  MOD_RES (23)..(23): 4-hydroxyproline
  MOD_RES (26)..(26): 4-hydroxyproline
  MOD_RES (29)..(29): 4-hydroxyproline
CGGPXGPXGPXGPXGPXGPXGPXGPXGPXGRGDSP